(19) United States
(12) Patent Application Publication
(10) Pub. No.: US 2017/0128599 A1
(43) Pub. Date: May 11, 2017

(54) FOLATE-CONJUGATES AND CORRESPONDING METAL-CHELATE COMPLEXES FOR USE IN DIAGNOSTIC IMAGING AND RADIOTHERAPY

(71) Applicant: MERCK & CIE, SCHAFFHAUSEN (CH)

(72) Inventors: Rudolf MOSER, Schaffhausen (CH); Roger SCHIBLI, Baden (CH); Cristina Magdalena MULLER, Nussbaumen (CH); Viola GROEHN, Dachsen (CH); Urs MICHEL, Wila (CH); Christoph SPARR, Appenzell (CH); Thomas Leighton MINDT, Basel (CH)

(73) Assignee: MERCK & CIE, SCHAFFHAUSEN (CH)

(21) Appl. No.: 15/358,787

(22) Filed: Nov. 22, 2016

Related U.S. Application Data
(63) Continuation of application No. 12/595,147, filed on Jan. 26, 2010, now abandoned.

(30) Foreign Application Priority Data
Apr. 11, 2007 (EP) 07105980.2

(51) Int. Cl. A61K 51/04; C07F 13/00; G01N 23/22; G01N 33/50; G01N 33/82; C07D 475/04; C07B 59/00

(52) U.S. Cl. CPC A61K 51/0459; C07D 475/04; C07F 13/00; C07B 59/002; G01N 33/5005; G01N 33/82; G01N 23/2204; C07B 2200/05; G01N 2333/705

(57) ABSTRACT

The present invention is directed towards novel folate-conjugates of formula I

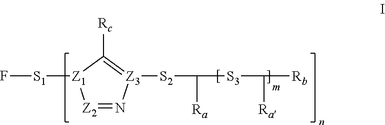

wherein F is a folate or derivative thereof, Z₁, Z₂, Z₃ are independently of each other C or N, S₁ to S₄ are independently of each other a single bond or a spacer, Rₐ, R_{a′}, and R_{b} are donor groups and/or another group F, R_{c} is optionally another group F and n is 1 or 2.

The invention further contemplates the corresponding metal-chelate complexes as well as pharmaceutical compositions thereof, and their uses in diagnostic imaging and radiotherapy.

### FIELD OF THE INVENTION

The present invention relates to novel folate-conjugates and the corresponding metal-chelate complexes as well as pharmaceutical compositions thereof, their method of production and their use in diagnostic and therapeutic medical applications, such as diagnostic imaging and radiotherapy.

### BACKGROUND OF THE INVENTION

Cell-specific targeting for delivery of diagnostic or therapeutic agents is a widely researched field and has led to the development of noninvasive diagnostic and/or therapeutic medical applications. In particular in the field of nuclear medicine procedures and treatments, which employ radioactive materials emitting electromagnetic radiations as gamma rays or photons, selective localization of these radioactive materials in targeted cells or tissues is required to achieve either high signal intensity for visualization of specific tissues, assessing a disease and/or monitoring effects of therapeutic treatments, or high radiation dose, for delivering adequate doses of ionizing radiation to a specified diseased site, without the risk of radiation injury in other tissues.

The folate receptor (FR) is a high-affinity membrane-associated protein, which exhibits limited expression on healthy cells, but is frequently overexpressed on a wide variety of specific cell types, such as epithelial tumor cells (e.g. ovarian, endometrial, breast, colorectal, kidney, lung, nasopharyngeal) and activated (but not resting) macrophages, which are involved in inflammation and autoimmune diseases. This led to the use of folic acid and its derivatives as a targeting agent for the delivery of pharmaceutical and/or diagnostic agents to these specific cell populations to achieve a selective concentration of pharmaceutical and/or diagnostic agents in these specific cells relative to normal cells. Such folate-conjugates include folate radiopharmaceuticals (Leamon and Low, Drug Discov. Today 2001; 6:44-51), folate-conjugates of chemotherapeutic agents (Leamon and Reddy, Adv. Drug Deliv. Rev. 2004; 56:1127-41; Leamon et al, Bioconjugate Chem. 2005; 16:803-11), proteins and protein toxins (Ward et al., J. Drug Target. 2000; 8:119-23; Leamon et al, J. Biol. Chem. 1993; 268:24847-54; Leamon and Low, J. Drug Target. 1994; 2:101-12), antisense oliconucleotides (Li et al, Pharm. Res. 1998; 15:1540-45; Zhao and Lee, Adv. Drug Deliv. Rev. 2004; 56:1193-204), liposomes (Lee and Low, Biochim. Biophys. ActaBiomembr. 1995; 1233:134-44); Gabizon et al, Adv. Drug Deliv. Rev. 2004; 56:1177-92), hapten molecules (Paulos et al, Adv. Drug Deliv. Rev. 2004; 56:1205-17); MRI contrast agents (Konda et al, Magn. Reson. Mat. Phys. Biol. Med. 2001; 12:104-13) etc.

Known folate radiopharmaceuticals include for example conjugates with ¹²⁵I-labeled histamine (U.S. Pat. No. 4,136,159), with small metalchelants such as deferoxamine (U.S. Pat. No. 5,688,488), acyclic or cyclic polyaminocarboxylates (e.g. DTPA, DTPA-BMA, DOTA and DO3A; U.S. Pat. No. 6,221,334), bisaminothiol (U.S. Pat. No. 5,919,934), 6-hydrazinonicotinamido-hydrazido (Shuang Liu, Topics in Current Chemistry, vol 252 (2005), Springer Berlin/Heidelberg), and ethylenedicysteine (U.S. Pat. No. 7,067,111), and small peptides (U.S. Pat. No. 7,128,893).

However, there is still a need for alternative, highly selective radionuclide conjugates, which can be synthesized easily and which exhibit optimal target (i.e. tumor cell, activated macrophage, etc.) to non-target tissue ratios and are eliminated through the kidneys, for use as tumor imaging agents in highly selective and non-invasive procedures permitting early detection and treatment of tumor cells, activated macrophages (and other targeted cells exhibiting high FR expression, not yet identified).

Applicants have now found novel folate-conjugates that are able to overcome the drawbacks of known conjugates and meet the current needs by showing several advantages, such as improved labeling efficiency at low ligand concentration, stable complex formation, better biodistribution, increased target tissue uptake and better clearance from non-targeted tissues and organs. These novel folate-conjugates comprise a chelating moiety and a pharmacological transport/binding moiety. The novel folate-conjugates can form a stable chelate with various radionuclides suitable for diagnostic imaging and radiotherapeutic applications. More specifically, the novel conjugates are based on five-membered heterocycles and designed such that the affinity of the pharmacological entity for its receptor is not compromised by the binding to at least one radionuclide.

### SUMMARY OF THE INVENTION

The present invention relates in a first aspect to novel folate-conjugates, hereinafter also called compounds of the invention, and their complexes with at least one radionuclide, which can overcome one or more of the disadvantages associated with the related art as discussed hereinabove.

In a specific embodiment the present invention is directed to a compound of formula I

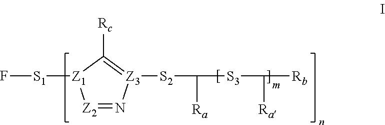

wherein
F is a folate or derivative thereof,
Z₁, Z₂, Z₃ are independently of each other C or N,
S₁, S₂, S₃ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C|C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or straight chain or branched C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group.
R_{c} is H, CO₂R′, COR′, —SO₃R′, —NHR′, wherein R′ represents H or C1-C6 alkyl, or straight chain or branched C1-C12 alkyl, which is unsubstituted or substituted by at least one CN, Hal, or NO₂, or a F as defined hereinabove,
m is 0, 1, 2, 3, or 4, and
n is 1 or 2

In one embodiment, the present invention is directed to a compound of formula I, wherein F is represented by a pteroyl-derivative as shown in a compound of formula II and II′

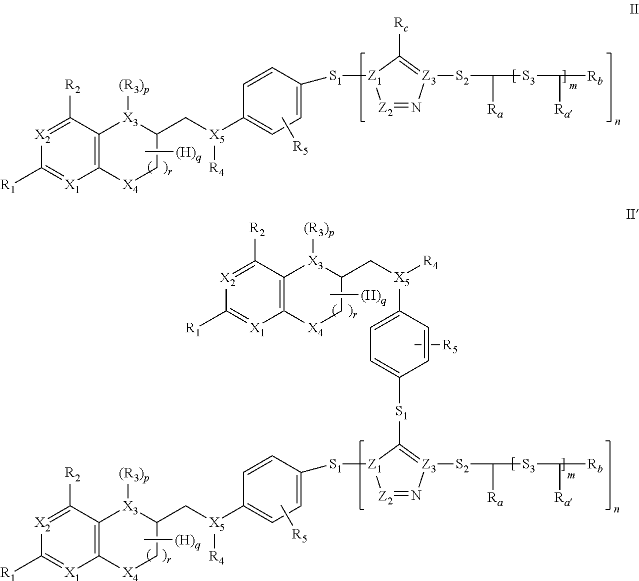

wherein
X₁, X₂, X₃, X₄ and X₅ are independently of each other C or N,
Z₁, Z₂, Z₃ are independently of each other C or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
S₁, S₂, S₃ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C|C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group,
R_{c} is H, CO₂R′, COR′, —SO₃R′, —NHR′, or straight-chain or branched C1-C12 alkyl, which is unsubstituted or substituted by at least one CN, Hal, or NO₂, wherein R′ represents H, C1-C6 alkyl,
m is 0, 1, 2. 3 or 4,
n is 1 or 2,
p has a value of 0, 1 or 2,
q has a value of 1 to 7, and
r is 0 or 1.

The scope of the present invention encompasses all possible permutations (shown or not shown) of groups Rₐ, R_{a′} and R_{b} being a group F within compounds of formula II and II′ as further described hereinafter.

In another embodiment, the present invention is directed to a compound of formula I, wherein F is represented by a folic acid (i.e. a pteroyl-glutamic acid) derivative as shown in formula III

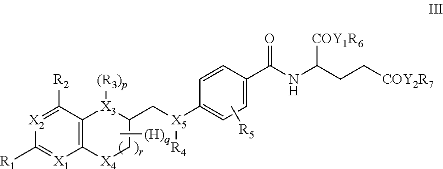

wherein
X₁, X₂, X₃, X₄ and X₅ are independently of each other C or N;
Y₁, Y₂ are independently of each other C, O or N,
R₁ to R₄ and p, q, and r are defined as hereinabove,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
R₆ and R₇ are independently of each other H, straight chain or branched C₁-C₁₂ alkyl, which is unsubstituted or substituted by at least one CN, Hal or NO₂, or a group of formula IV

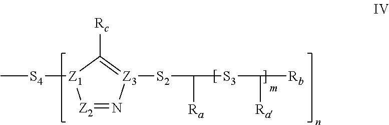

wherein
Z₁, Z₂, Z₃ are independently of each other C or N,
S₂, S₃, S₄ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group
R_{c} is H, CO₂R′, COR′, —SO₃R′, —NHR′, wherein R′ represents H or C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4, and
n is 1 or 2,
with the proviso that at least one of R₆ and R₇ is a group of formula IV.

In one preferred embodiment m is 0. In another preferred embodiment m is 1.

In another aspect, the invention provides complexes comprising compounds of the present invention and ^{99m}Tc, ^{186/188}Re, ^{11l}In⁺³, ^{67/68}Ga⁺³, ⁹⁰Y⁺³, ¹⁰⁹Pd⁺², ¹⁰⁵Rh⁺³, ¹⁷⁷Lu, ^{64/67}Cu ¹⁶⁶Ho, ²¹³Bi.

In a further aspect the present invention provides methods for synthesizing a compound of the invention and the corresponding metal-chelate complex thereof.

In yet a further aspect the invention provides pharmaceutical compositions comprising a diagnostic imaging amount or a therapeutically effective amount of at least one complex of the present invention and a pharmaceutically acceptable carrier therefor. In a preferred embodiment, the pharmaceutical compositions contain at least one complex that contains Tc-99m, Re-186 or Re-188.

In a further aspect the present invention provides uses of complexes and/or pharmaceutical compositions of the present invention for convenient and effective administration to a subject in need for diagnostic imaging or radiotherapy. The subject of the methods of the present invention is preferably a mammal, such as an animal or a human, preferably a human.

In a further aspect the present invention provides a single or multi-vial kit containing all of the components needed to prepare the compounds of this invention, other than the radionuclide ion itself.

Other features and advantages of the invention will be apparent from the following detailed description thereof and from the claims.

### BRIEF DESCRIPTION OF FIGURES

FIG. 1. Generalised synthesis of a compound of the invention of formula III (4) and complexes thereof (5), wherein Z₁ is N and Z₂ and Z₃ are C (LG represents a suitable leaving group and PG represents a suitable protecting group).

FIG. 2. Generalised synthesis of a compound of the invention of formula III (9) and complexes thereof (10), wherein Z₁ and Z₂ are N and Z₃ is C.

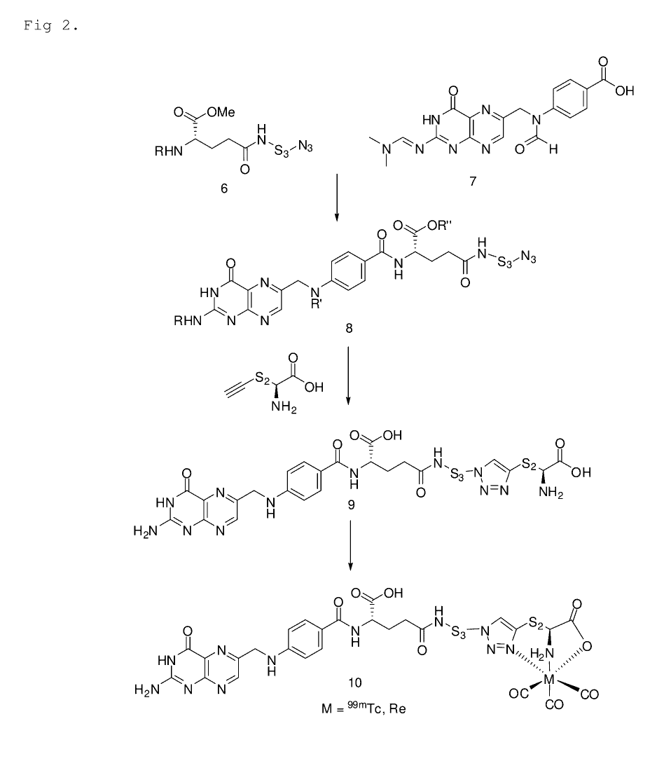

FIG. 3 (A) Biodistribution of ^{99m}Tc-His-Folate 4 h and 24 h p.i.; (B) Biodistribution of ^{99m}Tc-His-Folate 4 h p.i. with Pe-metrexed preinjected.

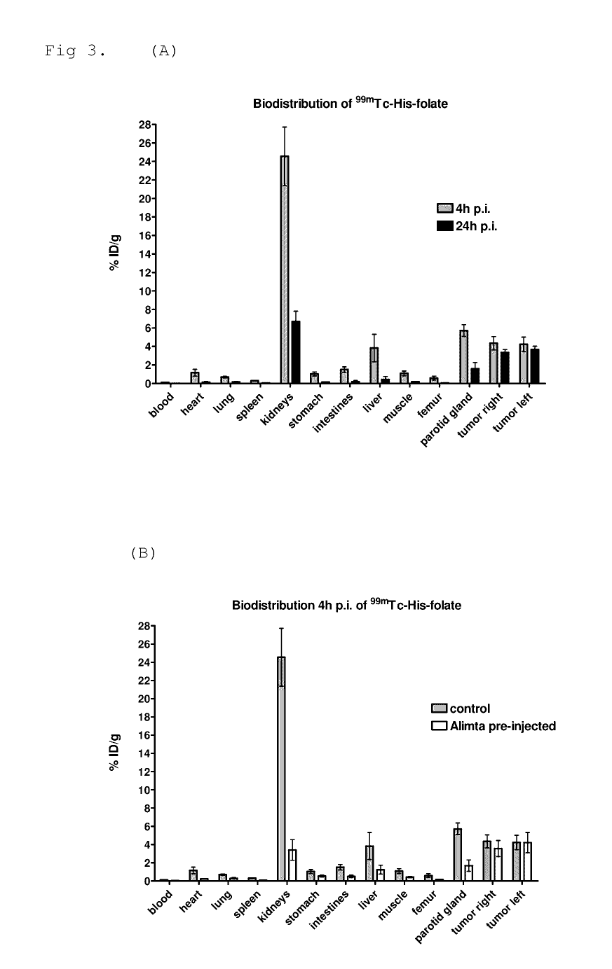

FIG. 4 (A) Biodistribution of ^{99m}Tc(CO)₃-Triazole-Folate 1 h, 4 h and 24 h p.i.; (B) Biodistribution of ^{99m}Tc(CO)₃-Triazole-Folate 4 h p.i. with Pe-metrexed preinjected.

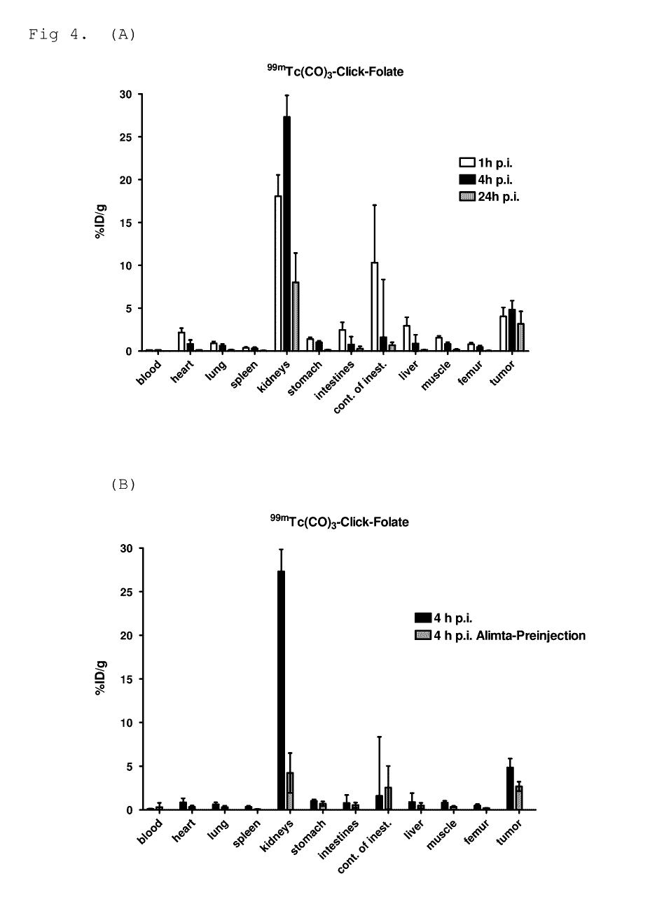

FIG. 5. (A) Ex vivo and (B) in vitro autoradiograms of KB Tumors and Kidneys using ^{99m}Tc-His-folate with or without Pe-metrexed.

FIG. 6. SPECT/CT-Picture of biodistribution studies in athymic nude mice using ^{99m}Tc-His-folate.

### DETAILED DESCRIPTION OF THE INVENTION

The present invention relates in a first aspect to novel folate-conjugates, hereinafter also called compounds of the invention, and their complexes with a radionuclide, which can overcome one or more of the disadvantages associated with the related art.

In a specific embodiment the present invention is directed to a compound of formula I

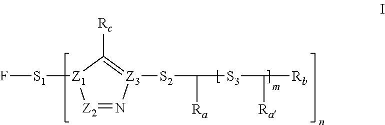

wherein
F is a folate or derivative thereof,
Z₁, Z₂, Z₃ are independently of each other C or N,
S₁, S₂, S₃ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl,

Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group

R_{c} is H, COOR′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4, and
n is 1 or 2.

A skilled person will know which permutations of compounds of formula I in combination with one or more groups F in position(s) Rₐ, R_{a′}, R_{b} (and R_{c}) can be part of this invention (as schematically illustrated in Scheme 1 with DG representing donor group):
(i) Some specific permutations include for example a compound of formula I, wherein m=0, i.e., wherein the only remaining groups Rₐ and R_{b} represent the two adjacent donor groups selected from —OH, —COOH, —NHR′, —CONH₂, —SH, a phosphine and a heterocyclic group and R_{c} may represent a group F.
(ii) Other permutations include for example a compound of formula I, wherein Rₐ and its neighbouring R_{a′} (i.e. m≧1) represent two adjacent donor groups selected from —OH, —COOH, —NHR′, —CONH₂, —SH, a phosphine and a heterocyclic group, and one or more of R_{a′} (for m>1), R_{b} and R_{c} may independently of each other represent a group F.
(iii) Further specific permutations include for example a compound of formula I, wherein R_{b} and its neighbouring R_{a′} (i.e. m 1) represent the two adjacent donor groups selected from —OH, —COOH, —NHR′, —CONH₂, —SH, a phosphine and a heterocyclic group and Rₐ, one or more of R_{a′} (for m>1), and R_{c} may independently of each other represent a group F.

Even further specific permutations (not shown in Scheme 1) include for example a compound of formula I, wherein two neighbouring R_{a′} groups (i.e. m≧2) represent the two adjacent donor groups selected from —OH, —COOH, —NHR′, —CONH₂, —SH, a phosphine, and a heterocyclic group and Rₐ, one or more of R_{a′} (for m>2), Rb and R_{c} may independently of each other represent a group F.

All of these permutations require the same coupling chemistries are thus they are all synthetically accessible to a skilled person. Thus it is understood that all of these possible permutations (shown or not shown) of compounds of formula I with a group F and two adjacent donor groups are part of this invention.

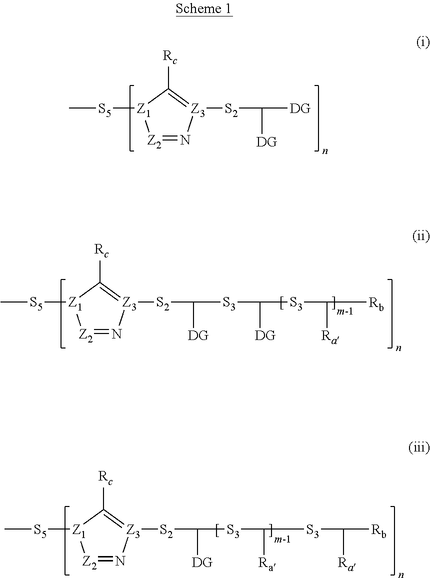

In a preferred embodiment a folate or derivative thereof, also hereinafter simply referred to as “a folate” or “folates”, for use in the present invention comprises compounds based on a condensed pyrimidine heterocycle, which is linked to linker S₁ (as defined hereinafter) through a benzoyl moiety. As used herein a “condensed pyrimidine heterocycle” includes a pyrimidine fused with a further 5- or 6-membered heterocycle, such as a pteridine or a pyrrolopyrimidine bicycle.

Preferred representatives of folates as used herein are based on a folate (pteroyl-glutamic acid) skeleton and include optionally substituted folic acid, folinic acid, pteropolyglutamic acid, and folate receptor-binding pteridines such as tetrahydropterins, dihydrofolates, tetrahydrofolates, and their deaza and dideaza analogs. Folic acid is the preferred conjugate-forming ligands used for the compounds of this invention. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5-dideaza, 5,10-dideaza, 8,10-dideaza, and 5,8-dideaza analogs. Preferred deaza analogs compounds include N-[4-[2-[(6R)-2-amino-1,4,5,6,7,8-hexahydro-4-oxopyrido[2,3-d]pyrimidin-6-yl]ethyl]benzoyl]-L-glutamic acid (Lometrexol) and N-[4-[1-[(2,4-diamino-6-pteridinyl)methyl]propyl]benzoyl]-L-glutamic acid (Edatrexate).

In a specific embodiment the present invention is directed to a compound of formula II and II′

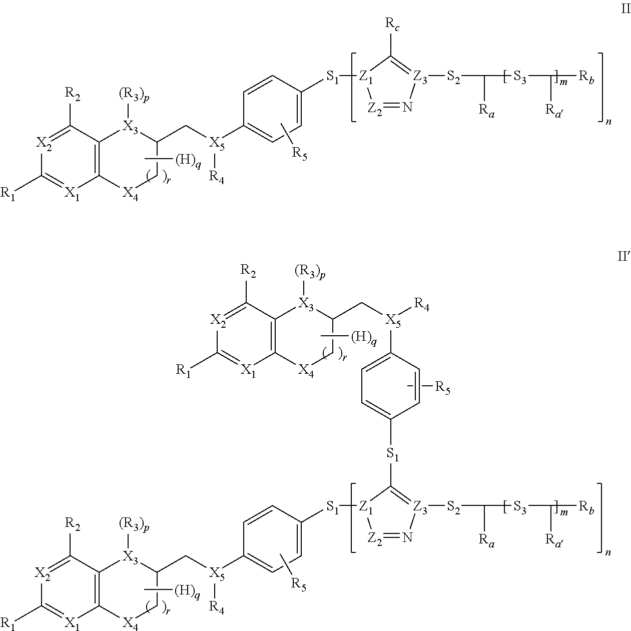

wherein
X₁, X₂, X₃, X₄ and X₅ are independently of each other C or N,
Z₁, Z₂, Z₃ are independently of each other C or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
S₁, S₂, S₃ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group
R_{c} is H, CO₂R′, COR′, —SO₃R′, —NHR′, or straight-chain or branched C1-C12 alkyl, which is unsubstituted or substituted by at least one CN, Hal, or NO₂, wherein R′ represents H, C1-C6 alkyl,
m is 0, 1, 2. 3 or 4,
n is 1 or 2,
p has a value of 0, 1 or 2,
q has a value of 1 to 7, and
r is 0 or 1.

It is understood that the scope of the present invention encompasses all possible permutations of groups Rₐ, R_{a′} and R_{b} being a group F as defined hereinabove within compounds of formula II and II′:

First, these permutations include a compound of formula II or

II′ having one further group F. These include a compound of formula II or II′, wherein (i) Rₐ is a group F, or (ii) R_{b} is a group F, or (iii) R_{a′} is a group F.

Second, these permutations further include a compound of formula II or II′ having two further groups F. These include a compound of formula II or II′, wherein (i) Rₐ and R_{a′} are a group F, or (ii) Rₐ and R_{b} are a group F, or (iii) R_{a′} and R_{b} are a group F.

It is understood that in all of these permutations “m” has to be adjusted such that the requirement of having two adjacent donor groups is still fulfilled.

Selected embodiments of the above described permutations according to the present invention are for example compounds of formulas IIa, IIb, IIc, IId and IIe

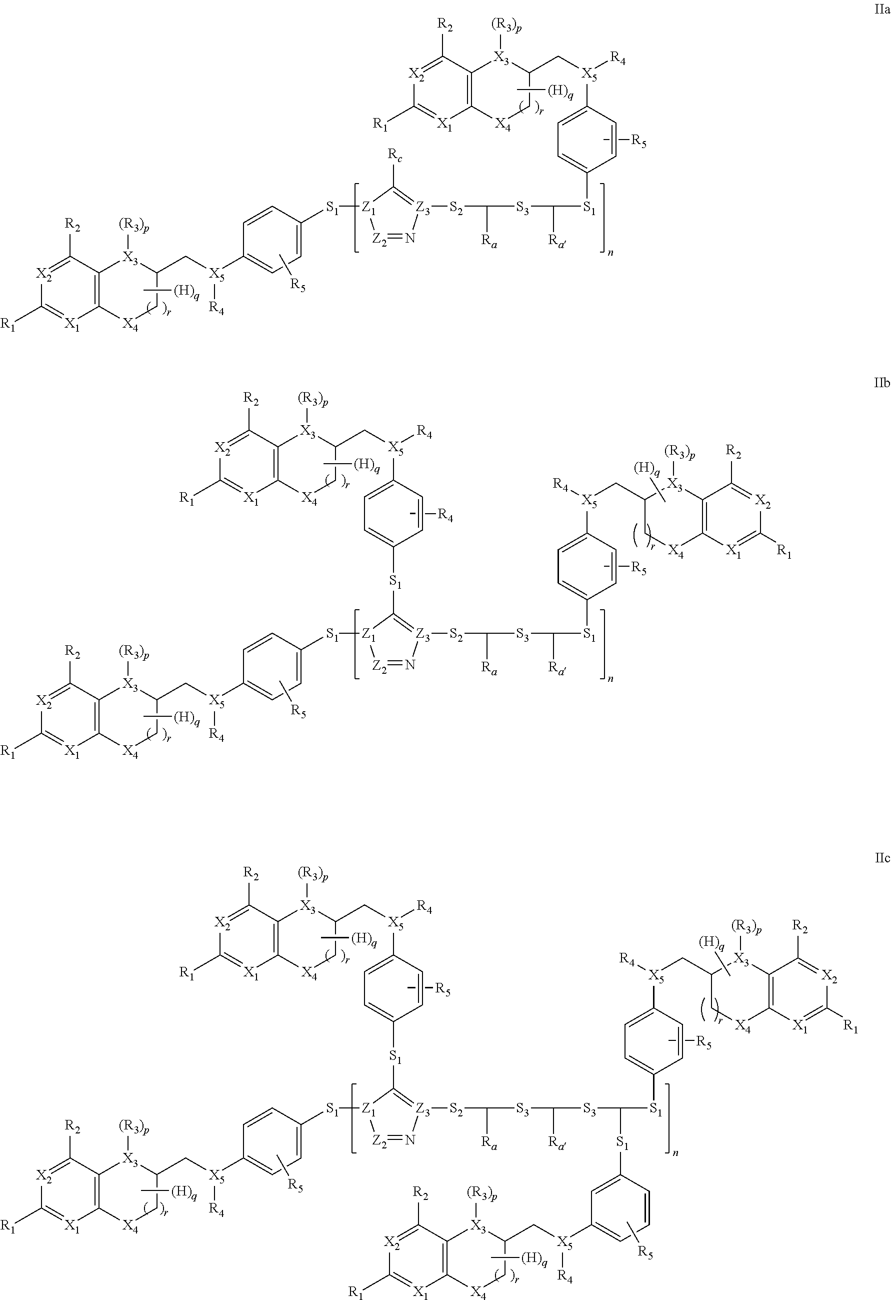

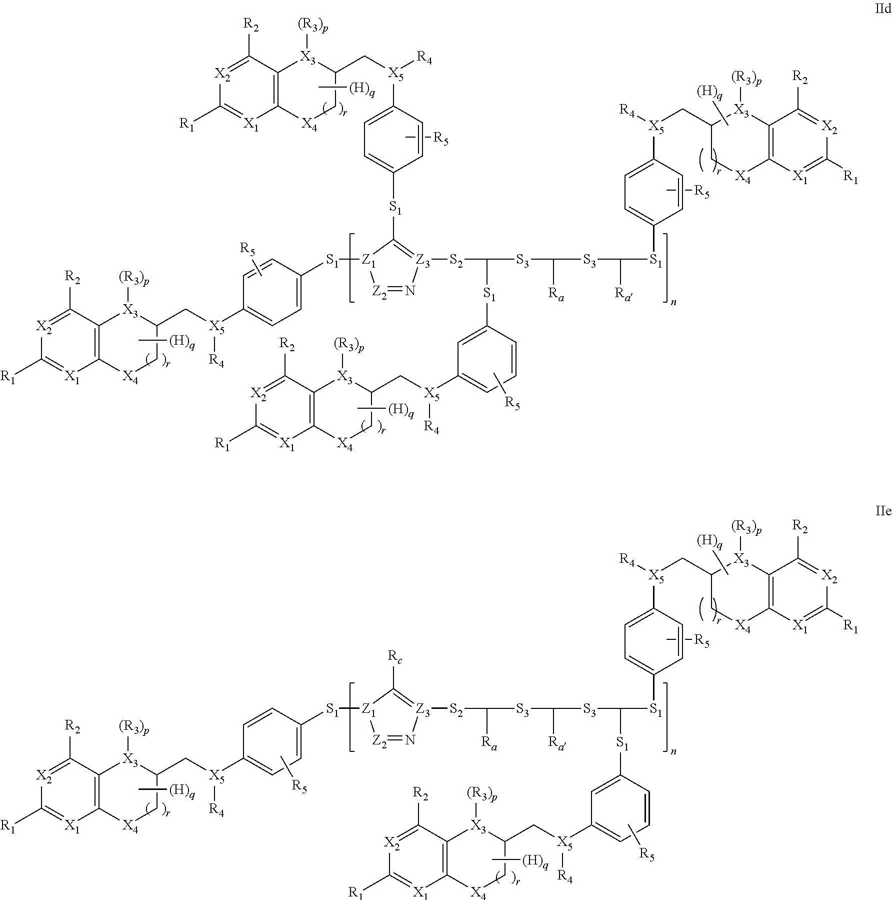

wherein
X₁, X₂, X₃, X₄ and X₅ are independently of each other C or N,
Z₁, Z₂, Z₃ are independently of each other C or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
S₁, S₂, S₃ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other —OH, —COOH, —NHR′, —CONH₂, —SH, a phosphine or a heterocyclic group, wherein R′ represents H, C1-C6 alkyl,
R_{c} is H, CO₂R′, COR′, —SO₃R′, —NHR′, or straight-chain or branched C1-C12 alkyl, which is unsubstituted or substituted by at least one CN, Hal, or NO₂, wherein R′ represents H, C1-C6 alkyl,
n is 1 or 2,
p has a value of 0, 1 or 2,
q has a value of 1 to 7, and
r is 0 or 1.

It is understood that the scope of the invention encompasses all possible permuatations of formulas II and IIa′ (shown or not shown).

In one embodiment Z₁ is N, Z₃ is C and Z₂ is C or N.

In another embodiment Z₁ is C and Z₂ and Z₃ are N.

S₁ is preferably a single bond or straight-chain or branched C1-C12 alkyl, which is unsubstituted or substituted by at least one CN, Hal, OH, NH₂, SH, SO₃H or NO₂, and wherein one or more of the non-adjacent CH2 groups may independently be replaced by —O—, —CO—, —CO—O—, —O—CO—, —NR′—, —NR′—CO—, —CO—NR′—, —CH═CH—, —C≡C—, or a five- or six-membered aromatic ring, which is unsubstituted or substituted with CN, Hal, NO2, COR′ or COOR′, wherein R′ represents H or C1-C6 alkyl, or a combination thereof.

More preferably S₁ is a single bond or straight-chain or branched C1-C12 alkyl, which is unsubstituted or substituted by at least one CN, Hal, or NO₂, and wherein one or more of non-adjacent CH₂ groups may independently be replaced by —O—, —CO—, —CO—O—, —NR′—, —NR′—CO—, —CO—NR′—, wherein R′ represents H or C1-C6 alkyl.

S₂, S₃ are independently of each other preferably a single bond or straight-chain or branched C1-C12 alkyl, which is unsubstituted or substituted by at least one CN, Hal, OH, NH₂ or NO₂, and wherein one or more of the non-adjacent CH₂ groups may independently be replaced by —O—, —CO—, —CO—O—, —NR′—, —NR′—CO—, —CO—NR′—, —CH═CH—, —C≡C—, wherein R′ represents H or C1-C6 alkyl.

More preferably S₂, S₃ are independently of each other straight-chain or branched C1-C8 alkyl, which is unsubstituted or substituted by at least one CN, Hal, or NO₂, and wherein one or more of the non-adjacent CH₂ groups may independently be replaced by —O—, —CO—, —CO—O—, —NR′—, —NR′—CO—, —CO—NR′—, wherein R′ represents H or C1-C6 alkyl, most preferably S₂, S₃ are independently of each other straight-chain or branched C1-C6 alkyl, which is unsubstituted or substituted by at least one CN, Hal, OH, or NO₂.

In one preferred embodiment m=0, in another preferred embodiment m=1.

In a further preferred embodiment R_{c} is H, CO₂R′, COR′, —NHR′ or unsubstituted C1-C6 alkyl, wherein R′ represents H or C1-C6 alkyl.

Preferred embodiments of S₁ and R_{c} include amino acids, short peptides, sugar molecules. A person skilled in the art would know how to choose.

Thus, in a further preferred embodiment the present invention is directed to a compound of formula II, wherein S₁ is an amino acid moiety, i.e. wherein F represents a folate structure comprising a pteroyl moiety linked to an amino acid moiety. As used herein the term “amino acid” includes compounds with both an amino group (e.g., NH₂ or NH₃⁺) and a carboxylic acid group (e.g., COOH or COO⁻). In a specific embodiment, the amino acid may be an α-amino acid, a β-amino acid, a D-amino acid or an L-amino acid. The amino acid may be a naturally occurring amino acid (e.g., alanine, valine, leucine, isoleucine, proline, phenylalanine, tryptophan, methionine, glycine, serine, threonine, cysteine, tyrosine, asparagine, glutamine, aspartic acid, glutamic acid, lysine, arginine, or histidine, etc.) or it may be a derivative thereof. Examples of derivatives include optionally substituted amino acids, e.g. having one or more substituents selected from CN, Hal, and/or NO₂. The amino acid may also include any other non-naturally occurring amino acids, such as e.g. norleucine, norvaline, L- or D-naphthalanine, ornithine, homoarginine and others well known in the peptide art (see for example in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference). Amino acids and amino acid analogs/derivatives can be purchased commercially (Sigma Chemical Co.; Advanced Chemtech) or synthesized using methods known in the art. In another specific embodiment, the amino acid may also be part of a polyamino acid (also termed polypeptide), wherein a plurality of same or different amino acids as defined hereinabove are covalently linked, i.e. linked through conventional peptide or other bonds.

Preferred amino acids include for example glutamic acid, aspartic acid, glutamine, aspartine, lysine, arginine, cystein, and derivatives thereof and preferred polyamino acids include homopolymers the respective homopolymers thereof (i.e. polyglutamic acid, polyaspartic acid, etc). Most preferred are optionally substituted aspartic and glutamic acid.

Thus in a more specific embodiment the present invention is directed to a compound of formula II, wherein F represents a pteroyl glutamic acid (or folic acid) skeleton having two attachment sites as represented by compound of formula III,

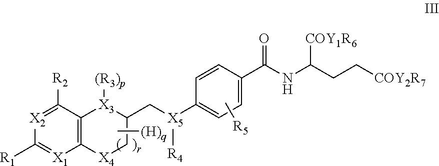

wherein
X₁, X₂, X₃, X₄ and X₅ are independently of each other C or N;
Y₁, Y₂ are independently of each other C, O or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
p has a value of 0, 1 or 2,
q has a value of 1 to 7,
r is 0 or 1,
R₆ and R₇ are independently of each other H, straight chain or branched C₁-C₁₂ alkyl, which is unsubstituted or substituted by at least one CN, Hal or NO₂, or a group of formula IV

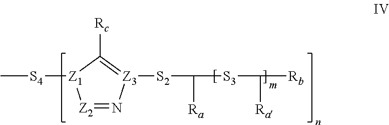

wherein
Z₁, Z₂, Z₃ are independently of each other C or N,
S₂, S₃, S₄ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group.
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4, and
n is 1 or 2,
with the proviso that at least one of R₆ and R₇ is a group of formula IV.

In a specific embodiment, either (i) Z₁ is N, Z₃ is C and Z₂ is C or N, or (ii) Z₁ is C and Z₂ and Z₃ are N.

In a preferred embodiment, S₂, S₃, S₄ are independently of each other a single bond or straight-chain or branched C1-C12 alkyl, which is unsubstituted or substituted by at least one CN, Hal, OH, NH₂ or NO₂, and wherein one or more of the non-adjacent CH₂ groups may independently be replaced by —O—, —CO—, —CO—O—, —NR′—, —NR′—CO—, —CO—NR′—, —CH═CH—, —C≡C—, wherein R′ represents H or C1-C6 alkyl.

More preferably S₂, S₃, S₄ are independently of each other straight-chain or branched C1-C8 alkyl, which is unsubstituted or substituted by at least one CN, Hal, or NO₂, and wherein one or more of the non-adjacent CH₂ groups may independently be replaced by —O—, —CO—, —CO—O—, —NR′—, —NR′—CO—, —CO—NR′—, wherein R′ represents H or C1-C6 alkyl.

Most preferably S₂, S₃, S₄ are independently of each other straight-chain or branched C1-C6 alkyl, which is unsubstituted or substituted by at least one CN, Hal, OH, or NO₂.

In a further preferred embodiment R_{c} is H, CO₂R′, COR′, —SO₃R′, —NHR′ or C1-C12 alkyl, wherein R′ represents H or C1-C6 alkyl. In a specific embodiment the present invention is directed to a compound of formula III, wherein (a) R₆ is H, straight chain or branched C₁-C₁₂ alkyl, which is unsubstituted or substituted by at least one CN, Hal or NO₂, and R₇ is a group of formula IV, (b) R₆ is a group of formula IV, and R₇ is H, straight chain or branched C₁-C₁₂ alkyl, which is unsubstituted or substituted by at least one CN, Hal or NO₂, or (c) both R₆ and R₇ are a group of formula IV.

In a further specific embodiment m is 0 or 1.

Thus the present invention is directed towards compounds of formula III, wherein at least one of R₆ and R₇ is a group of formula IVa, IVb and/or a group of formula IVb′

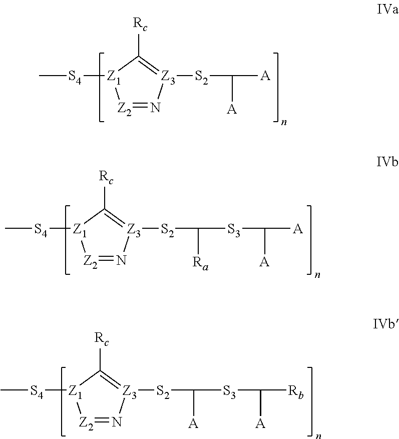

wherein
Z₁, Z₂, Z₃ are independently of each other C or N,
S₂, S₃, S₄ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
A represents independently of each other —COOH, —NH₂, —CONH₂, or —SH,
Rₐ, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove,
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove, and
n is 1 or 2.

It is understood that all possible permutations (shown or not shown) of compounds of formula III together with a group of formula IV in combination with a group F and two adjacent donor groups are part of this invention. These include a compound of formula III, wherein either R₆ or R₇ or both are a group of formula IV, and wherein Rₐ, R_{a′}, R_{b}, and/or R_{c} in each of these compounds may represent a group F (whereby two of Rₐ, R_{a′}, R_{b} are adjacent donor groups, as e.g. shown in the specific embodiment of group of formulae IVa, IVb and IVb′). Selected compounds are depicted by formulas V and V′, Va and Va′, Vb and Vb′, wherein the possibility of R_{c} being a group F is illustrated. It is understood that all other permutations with Rₐ, R_{a′} and R_{b} being a group F and which are not illustrated are also within the scope of invention.

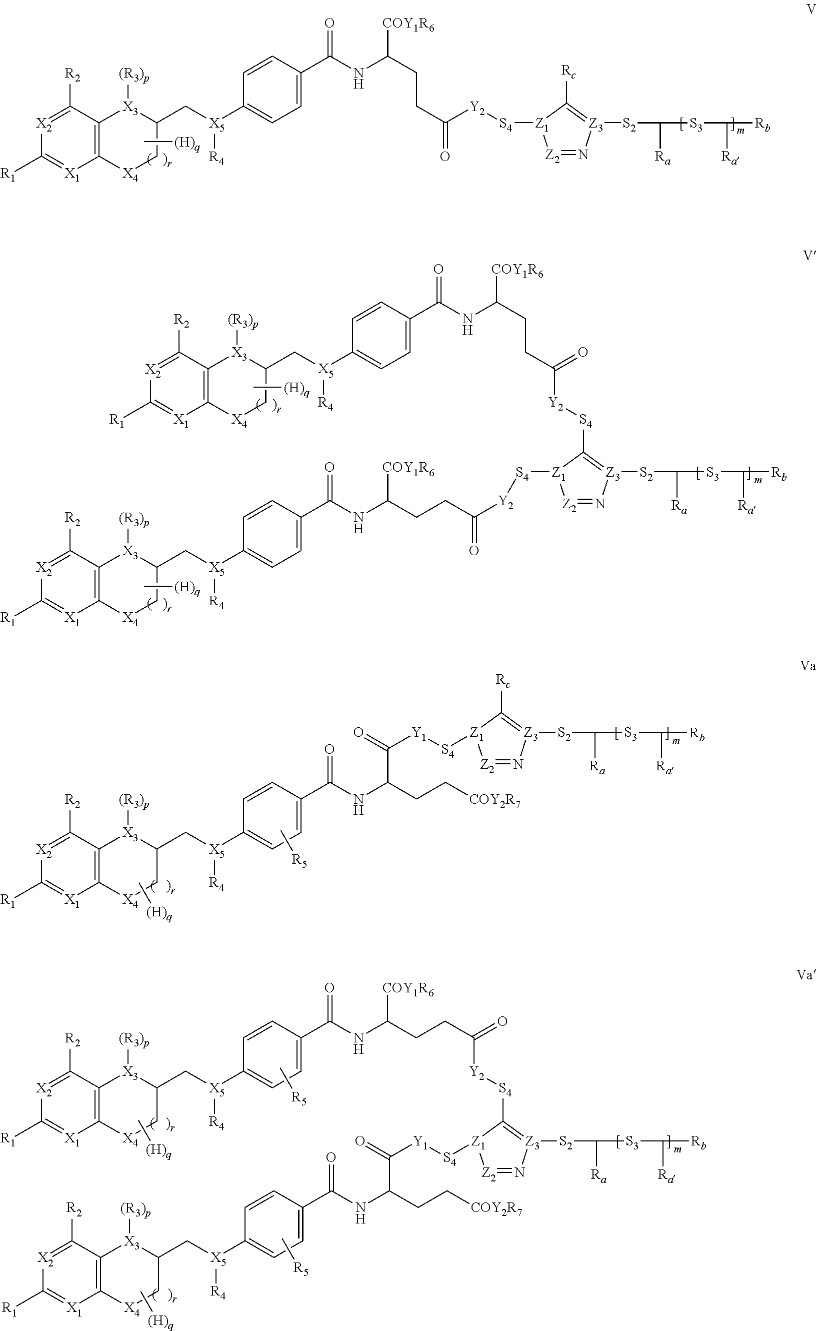

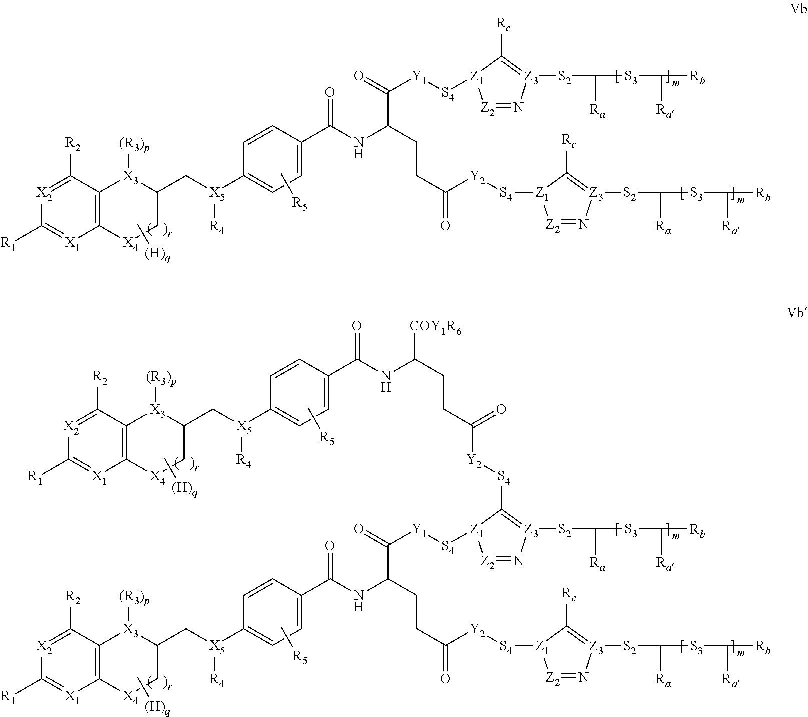

wherein
X₁, X₂, X₃, X₄ and X₅ are independently of each other C or N;
Y₁, Y₂ are independently of each other C, O or N,
Z₁, Z₂, Z₃ are independently of each other C or N;
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
R₆, R₇ are independently of each other H, straight chain or branched C₁-C₁₂ alkyl, which is unsubstituted or substituted by at least one CN, Hal or NO₂,
S₂, S₃, S₄ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4,
p has a value of 0, 1 or 2,
q has a value of 1 to 7, and
r is 0 or 1.

In a more preferred embodiment the present invention contemplates compounds wherein S₂, S₃, S₄ are independently of each other straight-chain or branched C1-C8 alkyl, which is unsubstituted or substituted by at least one CN, Hal, OH, or NO₂ and wherein one or more of non-adjacent CH₂ groups may independently be replaced by —O—, —CO—, —CO—O—, —NR′—, —NR′—CO—, —CO—NR′—, wherein R′ represents H or C1-C6 alkyl.

In a further specific embodiment m is 0 or 1.

Thus, in a further preferred embodiment the present invention is for example directed to a compound of formulas VI and VI′, VIa and VIa′, and VIb and VIb′,

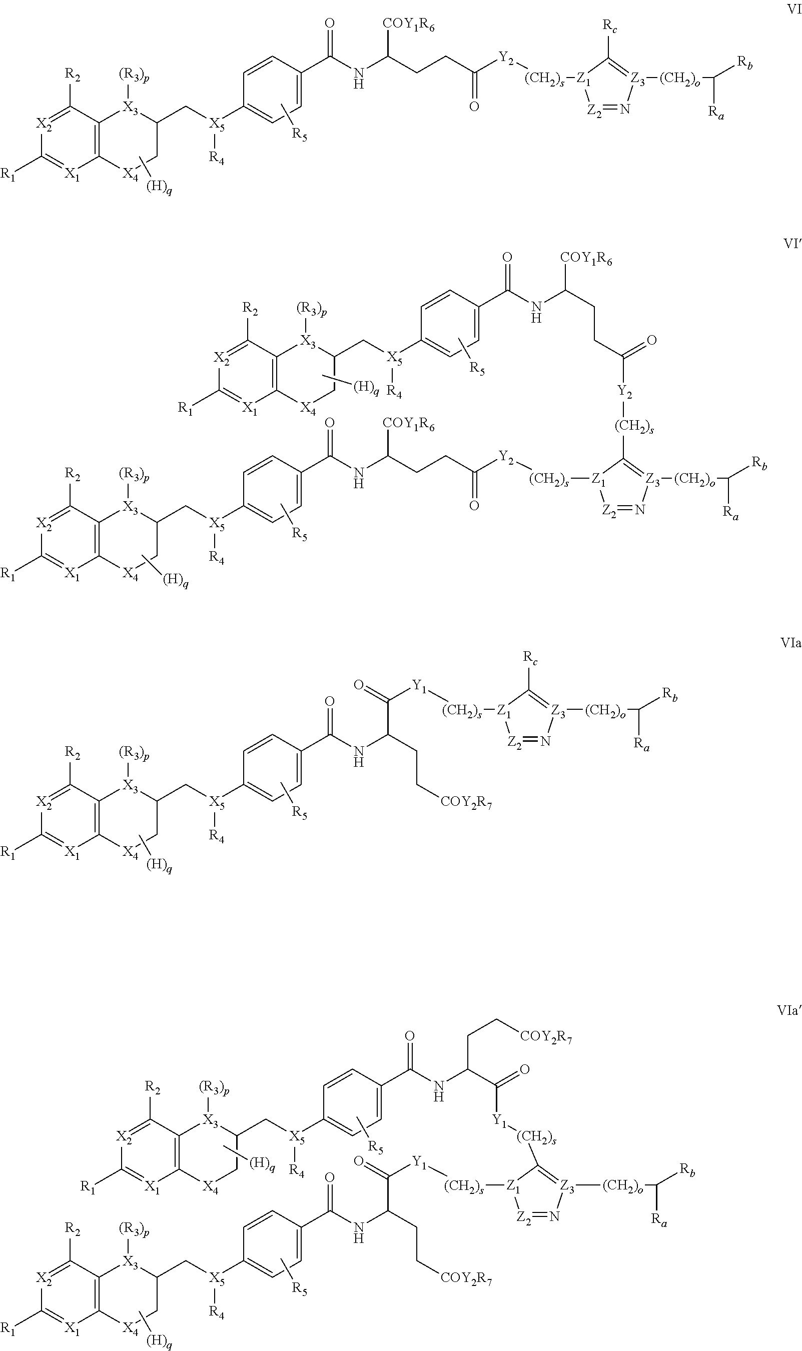

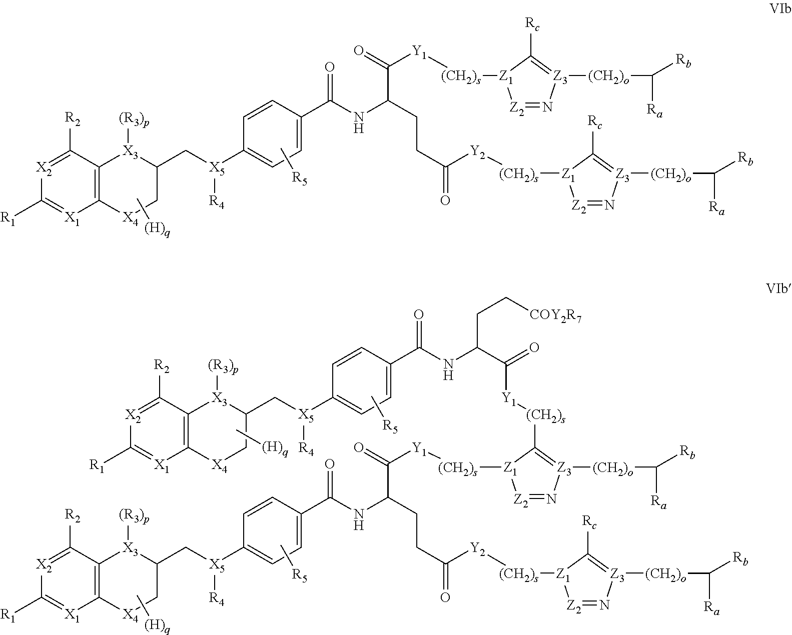

wherein
X₁, X₂, X₃, X₄ and X₅ are independently of each other N or C,
Z₁, Z₂, Z₃ are independently of each other C or N,
Y₁, Y₂ are independently of each other C, O or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
R₆, R₇ are independently of each other H or straight chain or branched C₁-C₁₂ alkyl, which is unsubstituted or substituted by at least one CN, Hal or NO₂,
Rₐ, R_{b} are independently of each other a donor group such as —OH, —COOH, —NHR′, —CONH₂, —SH, or a heterocyclic group selected from pyridyl, pyrrolyl, and thiazolyl, wherein R′ represents H or C1-C6 alkyl
R_{c} is H, CO₂R′, COR′, —SO₃R′, —NHR′, wherein R′ represents H or C1-C6 alkyl, or straight-chain or branched C1-C12 alkyl, which is unsubstituted or substituted by at least one CN, Hal, or NO₂,
p has a value of 0, 1 or 2,
q has a value of 1 to 7,
s is 1 to 8, and
o is 1 to 6.

Preferably either (i) Z₁ is N, Z₃ is C and Z₂ is C or N, or (ii) Z₁ is C and Z₂ and Z₃ are N.

Thus, in a further preferred embodiment the present invention is directed to a compound of formulae VII and VII′, VIIa and VIIa′, and VIIb and VIIb′

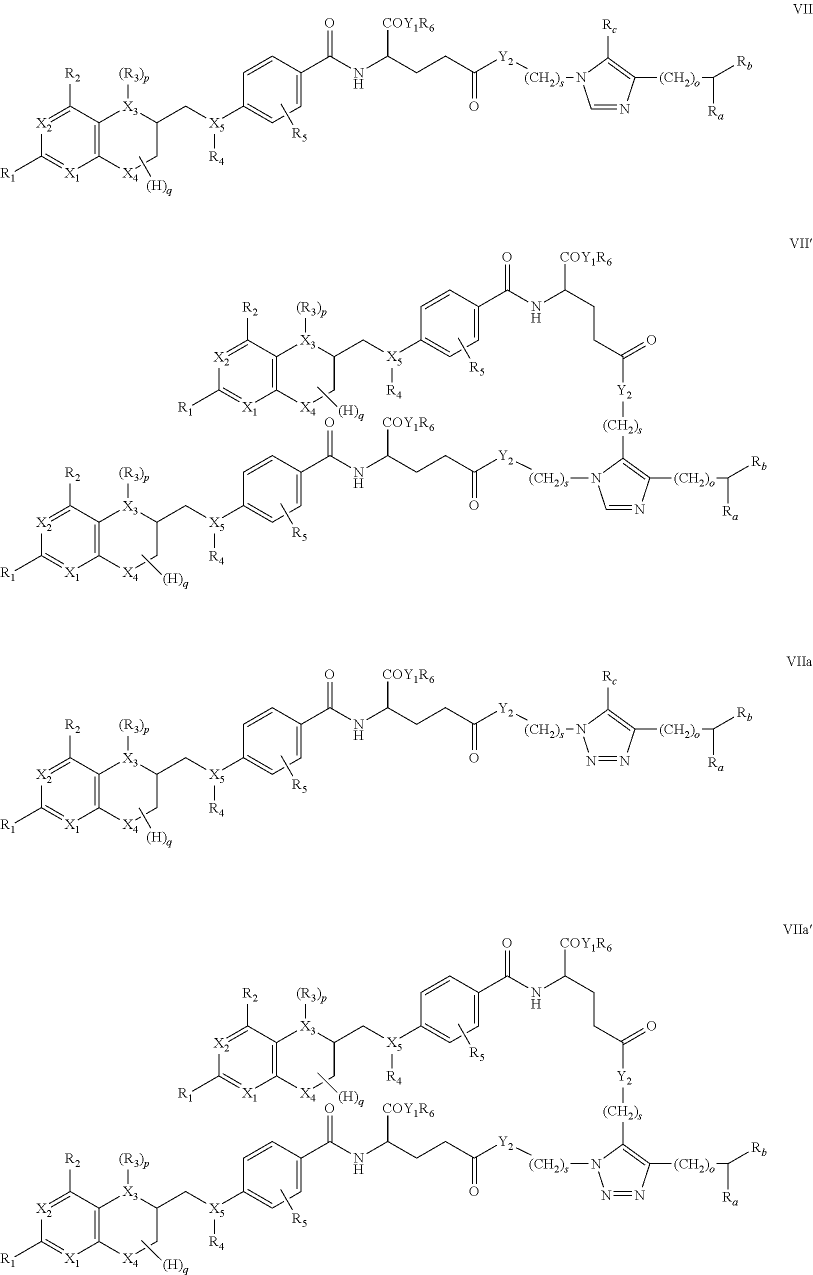

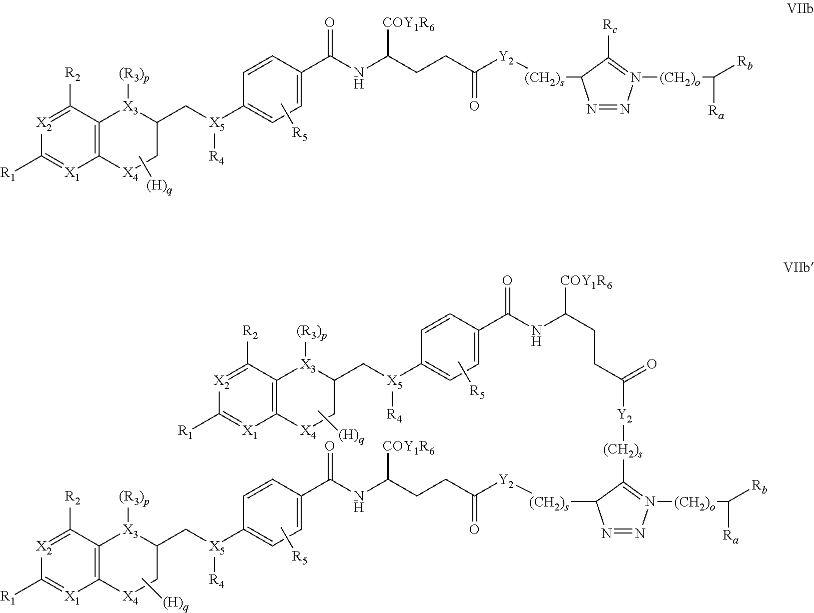

wherein
X₁, X₂, X₃, X₄ and X₅ are independently of each other N or C,
Y₁, Y₂ are independently of each other C, O or N,
R₁ and R₂ are independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl, wherein R′ is H or C1-C6 alkyl,
R₃ and R₄ are independently of each other H, formyl, iminomethyl, nitroso, C1-C12 alkyl, C1-C12 alkoxy, C1-C12 alkanoyl, halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
R₆ is H or straight chain or branched C₁-C₁₂ alkyl, which is unsubstituted or substituted by at least one CN, Hal or NO₂,
Rₐ and R_{b} are independently of each other a donor group such as —OH, —COOH, —NHR′, —CONH₂, —SH, or a heterocyclic group selected from pyridyl, pyrrolyl, and thiazolyl, wherein R′ represents H or C1-C6 alkyl
R_{c} is H, CO₂R′, COR′, —SO₃R′, —NHR′, wherein R′ represents H or C1-C6 alkyl, or straight-chain or branched C1-C12 alkyl, which is unsubstituted or substituted by at least one CN, Hal, or NO₂,
p has a value of 0, 1 or 2,
q has a value of 1 to 7,
s is 1 to 8, and
o is 1 to 6.

In a further preferred embodiment R_{c} is H, CO₂R′, COR′, —SO₃R′, —NHR′, wherein R′ represents H or C1-C6 alkyl, or C1-C12 alkyl.

In a most preferred embodiment Rₐ is —NH₂, R_{b} is —OH and R_{c} is H.

Preferably o is 1, 2, 3 or 4.

It is understood that the abbreviations “N” and “C” are representative for all possible degrees of saturation, i.e. N includes —NH— and —N═ linkages and C includes —CH₂— and —CH═ linkages.

It is understood that the abbreviation (H)_{q} represents all H substituents on the indicated ring (i.e. on X₃, C₆, C₇ and X₄). For example q=5 for a fully saturated unsubstituted analog (X₃=X₄=N, p=0) or q=7 for a fully saturated unsubstituted 5,8-dideaza analog (X₃=X₄=C, p=0) and q=1 for a fully unsaturated analog with X₃=X₄=N, p=0.

In a preferred embodiment, R₁ and R₂ may independently of each other represent H, alkyl, —OR′, —NHR′, more preferably —OR′, —NHR′.

In a preferred embodiment, R₃ is H, formyl, C1-C12 alkyl or C1-C12 alkanoyl.

In another preferred embodiment, R₄ is H, nitroso, C1-C12 alkoxy, or C1-C12 alkanoyl.

In a preferred embodiment, R₆ is H or straight chain or branched C₁-C₁₂ alkyl, which is unsubstituted or substituted by at least one CN, Hal or NO₂, more preferably R₆ is H or straight chain or branched C₁-C₁₂ alkyl. In a most preferred embodiment, R₆ is H.

In another preferred embodiment Rₐ, R_{a′}, R_{b} are independently of each other H, —OR′, —COOR′, —NHR′, —CONHR′, —SR′, or a heterocyclic group selected from pyridyl, pyrrolyl, and thiazolyl wherein R′ represents H or C1-C6 alkyl, or a F as defined hereinabove. More preferably Rₐ, R_{a′}, R_{b} are independently of each other H, —OR′, —COOR′, —NHR′, —CONHR′, —SR′, wherein R′ represents H or C1-C6 alkyl, or a F as defined hereinabove.

Preferred donor groups for Rₐ, R_{a′}, R_{b} are —OH, —COOH, —NHR′, —CONH₂, —SH, or a heterocyclic group selected from pyridyl, pyrrolyl, and thiazolyl, wherein R′ represents H or C1-C6 alkyl. More preferred donor groups for Rₐ, R_{a′}, R_{b} are independently of each other —OH, —COOH, —NHR′, —CONH₂, —SH, wherein R′ represents H or C1-C6 alkyl.

Further preferred embodiments include:
(i) X₁ to X₅ are N, R₁ is NH₂, R₂ is O, R₄ is H, s is 1, 3 or 5, and all the other parameters are as defined in formulae VII, VIIa or VIIb
(ii) X₁ to X₅ are N, Y is O, R₁ is NH₂, R₂ is O, R₃ is H, methyl or formyl, R₄ is H, methyl or formyl, R₆ is H, methyl or ethyl, s is 1, 3 or 5, and all the other parameters are as defined in formulae VII, VIIa or VIIb
(iii) X₁ to X₅ are N, Y is O, R₁ is NH₂, R₂ is O, R₃ is H, methyl or formyl, R₄ is H, R₆ is H, s is 1, 3 or 5, Rₐ and R_{b} are —OH, and all the other parameters are as defined in formulae VII, VIIa or VIIb

Thus, in a further specific embodiment the present invention is directed to a compound of formula VIII,

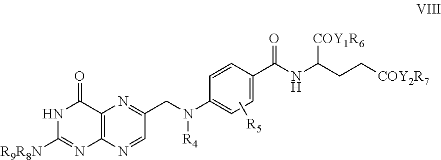

wherein,
Y₁, Y₂ are independently of each other C, N or O,
R₈, R₉ are independently of each other H, formyl, straight chain or branched C₁-C₁₂ alkyl, which is unsubstituted or substituted by at least one CN, Hal or NO₂,
R₄ is selected from H, nitroso, C1-C12 alkyl, C1-C12 alkoxy, C1-C12 alkanoyl, halosubstituted C1-C12 alkanoyl, and
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, and (C1-C12 alkylamino)carbonyl,
R₆ and R₇ are independently of each other H, straight chain or branched C₁-C₁₂ alkyl, which is unsubstituted or substituted by at least one CN, Hal or NO₂, or a group of formula IVa, IVb and/or a group of formula IVb′

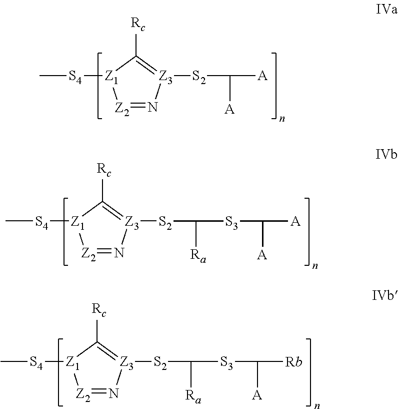

wherein
Z₁, Z₂, Z₃ are independently of each other C or N,
S₂, S₃, S₄ are independently of each other a single bond or a spacer, such as straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with CN, Hal, NO₂, COR′ or COOR′, wherein R′ represents H or C1-C6 alkyl,
A represents independently of each other —COOH, —NH₂, —CONH₂, or —SH,
Rₐ, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove,
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove, and
n is 1 or 2.

Preferably either (i) Z₁ is N, Z₃ is C and Z₂ is C or N, or (ii) Z₁ is C and Z₂ and Z₃ are N.

The term “alkyl”, when used singly or in combination, refers to straight chain or branched alkyl groups containing 1 to 12 carbon atoms, such as methyl, ethyl, propyl, isopropyl, butyl, secbutyl, isobutyl, t-butyl, pentyl isopentyl, neopentyl, hexyl and the like. The preferred alkyl groups contain 1 to 8, more preferably 1 to 4 carbon atoms.

As used herein, the term “alkenyl”, singly or in combination with other groups, refers to straight chain or branched alkylene groups containing 2 to 12 carbon atoms, such as methylene, ethylene, propylene, isopropylene, butylene, t-butylene, secbutylene, isobutylene, amylene, isoamylene, pentylene, isopentylene, hexylene and the like. The preferred alkenyl groups contain 2 to 6 carbon atoms.

The term “alkynyl” as used herein refers to a linear or branched chain of carbon atoms with one or more carbon-carbon triple bonds. The preferred alkynyl groups contain 2 to 12, more preferably 2 to 6 carbon atoms.

The term “alkoxy” as used herein refers to alkyl, as defined above, substituted with oxygen, such as methoxy, ethoxy, propoxy, isopropoxy, butoxy, tert-butoxy and the like.

The term “alkanoyl” as used herein refers to formyl, or alkyl, as defined above, terminally-substituted with a carbonyl such as acetyl, propanoyl, butanoyl, pentanoyl and the like.

The term “alkylamino” as used herein refers to alkyl, as defined above, substituted with nitrogen, including both monoalkylamino such as methylamino, ethylamino, propylamino, tert-butylamino, and the like, and dialkylamino such as dimethylamino, diethylamino, methylpropylamino, and the like.

The term “halo” as used herein refers to any Group 7 element and includes fluoro, chloro, bromo, iodo, and astatine(o).

The term “five- or six-membered aromatic carbocyclic or heterocyclic ring” refers to five- or six-membered aromatic carbocyclic rings such as phenyl, cycloheptyl, cyclohexyl, and cyclopentyl, and five- or six-membered aromatic heterocyclic rings containing at least one heteroatom selected from N, S, O, and P, such as pyridyl, piperidino, piperazino, morpholino, imidazolyl, triazolyl, tetrazolyl, oxazolyl, thiazolyl, pyrrolidinyl, and pyrazolyl.

The term “heterocyclic group” as used herein refers to a saturated heterocyclic group or unsaturated heterocyclic group having at least one heteroatom selected from N, S, O, and P, preferably N or S. Examples of a saturated heterocyclic group include tetrahydrofuryl, pyrrolidinyl, pyrazolidinyl, imidazolidinyl, piperidyl, morpholinyl, thiamorpholinyl and piperazinyl. Examples of a unsaturated heterocyclic group include furyl, pyrrolyl, thienyl, thiazolyl, isothiazolyl, oxazolyl, isooxazolyl and pyrazolyl, pyridyl, pyrimidinyl, pyrazinyl and pyridazinyl.

These heterocyclic groups may be substituted by alkyl such as methyl or ethyl, halogen atom or phenyl. When the heterocyclic group is substituted by phenyl, it may form a condensed ring combining the two adjacent carbon atoms in the heterocyclic group with the phenyl group. Examples of the condensed rings include benzothiazolyl, benzofuryl, quinazolinyl and quinoxalinyl. Preferred heterocyclic groups are pyridyl, pyrrolyl, and thiazolyl.

As used herein, the term “phosphine” includes, for example, triarylphosphines, trialkylphosphines and tris(dialkylamino)phosphines, which may have a substituent, and the like. Specific examples thereof include, for example, 1,2-bis(dimethylphosphino)ethane and tris-hydroxymethylenephosphine.

As used herein, the term “optionally substituted” includes C(1-6)alkyl, C(1-6)alkenyl, C(1-6)acyl, aryl C(1-6)alkylaryl, cyano, nitro and halo, preferably C(1-6)alkyl, cyano, nitro and halo, most preferably C(1-4)alkyl.

In another aspect, the invention provides complexes comprising compounds of the present invention and ^{99m}Tc, ^{186/188}Re, ¹¹¹In⁺³, ^{67/68}Ga⁺³, ⁹⁰Y⁺³, ¹⁰⁹Pd⁺², ¹⁰⁵Rh⁺³, ¹⁷⁷Lu, ^{64/67}Cu ¹⁶⁶Ho, ²¹³Bi hereinafter also called complexes of the present invention. Preferably the complexes of the present invention comprise ^{99m}Tc, ¹⁸⁶Re or ¹⁸⁶Re. Technetium, which is particularly useful as a diagnostic imaging agent, is preferably one or more of the radionuclides ^{99m}Tc, ^{94m}Tc or ⁹⁶Tc. As indicated hereinabove, the preferred radioisotope for medical imaging is ^{99m}Tc. Its 140 keV .gamma.-photon is ideal for use with widely-available gamma cameras. It has short (6 hour) half-life, which is desirable when considering patient dosimetry. Rhenium, which is particularly useful as a radiotherapy agent, is preferably one of the radionuclides ¹⁸⁶Re or ¹⁸⁸Re, or a mixture thereof.

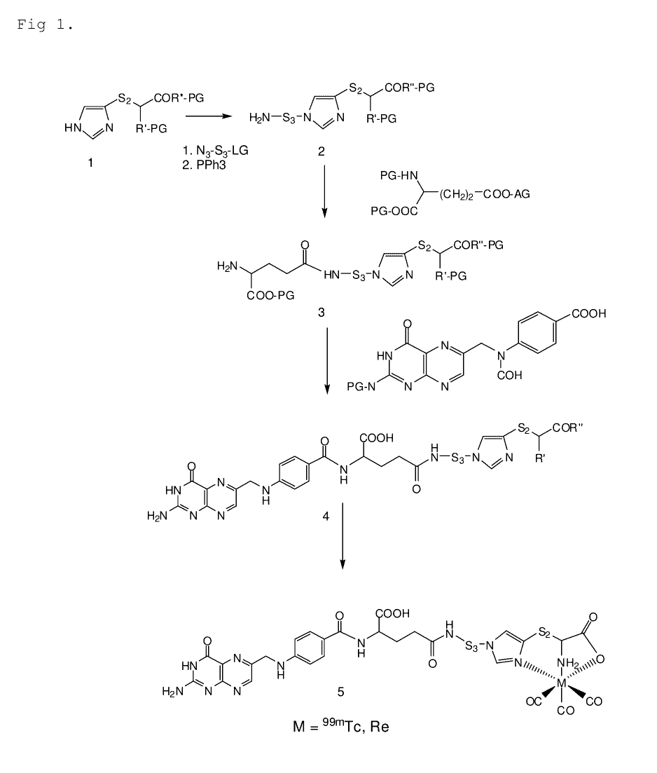

In a further aspect the present invention also provides methods of synthesizing a compound of the invention. In a first method of synthesis the heterocyclic ligand site for the radionuclide is synthesized first and subsequently linked through a suitable linker to a suitably protected pteroic or folic acid derivative to obtain the final compound of choice (see for example FIG. 1).

In one specific embodiment this method of synthesis for example includes in a first step coupling of a histidine fragment (compound 1 in FIG. 1), which is suitably protected at the amino- and carboxy-terminus to a linker S₃ having a suitable leaving group LG. Subsequent couplings to first a glutamic acid residue and then to a folic acid residue results in the final folic acid conjugate which upon deprotection can be complexed with a suitable radionuclide.

It will be obvious for a skilled person to select appropriate conditions for the various coupling steps and choose appropriate protecting groups PG (e.g. see Greene & Wuts, Eds., Protective Groups in Organic Synthesis, 2nd Ed., 1991, John Wiley & Sons, NY.) and leaving groups LG (e.g. a halogen, tosylate, mesylate, triflate, carbonate group).

In a second method of synthesis, a folic or pteroic acid moiety and a ligand site moiety are synthesized first, wherein the folic or pteroic acid moiety carries an azide group and the ligand site moiety carries an alkyne group (or vice versa) and subsequently coupled in a cycloaddition under thermal conditions or in the presence of a catalyst to obtain the final compound of choice (Kolb and Sharpless, Drug Discovery Today 2003, 8, 1128; Kolb et al. Angew. Chem. Int. Ed. 2001, 40, 2004; Rostovtsev, V. V. et al. Angew. Chem. Int. Ed. 2002, 41, 2596; US 2005/02222427; WO 06/116629).

These reactions are known as Huisgen 1,3-dipolar cycloaddition (thermal conditions) and Click-Reaction (catalytic conditions) and have been described in the art (Kolb and Sharpless, Drug Discovery Today 2003, 8, 1128; Kolb et al. Angew. Chem. Int. Ed. 2001, 40, 2004; Rostovtsev et al. Angew. Chem. Int. Ed. 2002, 41, 2596; US 2005/02222427; WO 06/116629). More specifically compounds of formula I wherein the five membered heterocycle is a triazole are obtained by cycloaddition of an azide Rₐ—N₃ with an alkyne R_{b}—C≡C—R_{c} and compounds of formula I wherein the five membered heterocycle is a tetrazole are obtained by cycloaddition of an azide Rₐ—N₃ with a cyanide R_{b}—C≡N. All possible combinations are contemplated herein, i.e. Rₐ being the folate derivative and R_{b} being a chelating moiety or precursor thereof as well as R_{b} being the folate derivative and Rₐ being a chelating moiety or precursor thereof. Thus the modular and versatile nature of the reaction allows to employ a wide variety of linkers to couple the radioisotope to folic acid.

In one specific embodiment the cycloaddition is performed under thermal conditions, i.e. at temperatures ranging from 10 to 200° C., preferably from 10 to 100° C.

In another embodiment the cycloaddition is performed in the presence of a catalyst, such as a transition metal complex, such as Ru and Cu(I). Preferred catalysts are Cu(I) salts, such as Cu(I) chloride, bromide, iodide. Alternatively Cu(I) can be obtained by in situ reduction of a Cu(II) salt. This reaction can be performed in a variety of protic or aprotic solvents, such as for example methanol, ethanol, 2-propanol, tertiary-butanol, n-butanol and/or water or buffered solutions thereof, at a wide range of temperatures (such as between 10 and 100° C., preferably room temperature) and varying pH (such as from 4 to 12), under oxidative or reducing conditions and in the presence of other functional groups with no need for protecting groups.

It will be obvious for a skilled person to select appropriate conditions (see also US 2005/0222427 which is incorporated herein by reference as well as references cited therein).

Thus, in one exemplary reaction, an alkynyl derivatized chelating moiety or precursor thereof (e.g. propargyl glycine) is coupled with azido folic acid under standard conditions (for example Na-ascorbate, Cu(OAc)₂, ^{t}BuOH/H₂O (1:1), rt). Alternatively, a chelating moiety or precursor thereof functionalized with an azido group is coupled to an alkyne substituted folic acid or derivative of choice in a Cu(I)-catalyzed cycloaddition under standard conditions. Both click product are then labelled with e.g. [^{99m}Tc(CO)₃(H₂O)₃]⁺ to provide the SPECT tracer.

Clearly, both routes allow the incorporation of a wide varieties of linkers to couple a different chealting moieties (and hence radiometals) to folic acid.

Specific embodiments are discussed in the Examples section.

The present invention further provides a method of synthesizing a complex of the invention, which comprises labeling a compound of the invention, which includes the steps of first obtaining a compound of the invention, and reacting the compound with a radionuclide as specified hereinabove, preferably Tc-99m, Re-186 or Re-188, generally in the presence of a reducing agent to form a metal chelate complex between the compound of the invention and the radionuclide. The reducing agent may be any known reducing agent, but will preferably be a dithionite ion or a stannous ion. In a specific embodiment, preparation of a complex of the present invention containing rhenium as the metal may be accomplished using rhenium in the +5 or +7 oxidation state. Examples of compounds in which rhenium is in the Re(VII) state are NaReO₄ or KReO₄. Re(V) is available as Re-gluconate, Re-glucoheptonate, Re-tartrate, Re-citrate. Other rhenium reagents capable of forming a rhenium complex may also be used.

In a further aspect the invention provides pharmaceutical compositions comprising a diagnostic imaging amount or a therapeutically effective amount of at least one complex of the present invention and a pharmaceutically acceptable carrier therefor. In a preferred embodiment, the pharmaceutical compositions contain at least one complex that contains Tc-99m, Re-186 or Re-188.

As used herein, a pharmaceutically acceptable carrier, which is present in an appropriate dosage, includes solvents, dispersion media, antibacterial and antifungal agents, isotonic agents, and the like which are physiologically acceptable. The use of such media and agents are well-known in the art.

In a further aspect the present invention provides uses of complexes and/or pharmaceutical compositions of the present invention for convenient and effective administration to a subject in need for diagnostic imaging or radiotherapy. The subject of the methods of the present invention is preferably a mammal, such as an animal or a human, preferably a human.

Thus in a particular embodiment the present invention provides a method for diagnostic imaging of a cell or population of cells expressing a folate-receptor, said method comprising the steps of administering at least one complex or composition of the present invention in a diagnostic imaging amount, and obtaining a diagnostic image of said cell or population of cells.

The complexes and/or compositions of the present invention may also be used as radiotherapy agents useful for the treatment of a subject in need thereof.

Thus in another particular embodiment the present invention provides a method for radiotherapy comprising the steps of administering to a subject in need thereof at least one complex or composition of the present invention in therapeutically effective amounts, and after localization of said at least one complex or composition in the desired tissues, subjecting the tissues to irradiation to achieve the desired therapeutic effect.

In yet another embodiment the present invention provides a method for simultaneous diagnosis and radiotherapy comprising the steps of administering to a subject in need thereof at least one complex or composition of the present invention in a diagnostically and therapeutically effective amount, and after localization of said at least one complex or composition in the desired tissues, subjecting the tissues to irradiation, and obtaining a diagnostic image of said tissues to follow the course of treatment.

An image of a cell or tissue expressing the folate receptor, i.e. a tumor cell or tissue, labeled with one or more of the complexes or compositions of the present invention can be detected using a radiation detector, e.g. a γ-radiation detector. One such procedure utilizes scintigraphy. Tomographic imaging procedures such as single photon emission computed tomography (SPECT) can also be used to improve visualization. Selection and use of such radiation detectors is within the skill of one of ordinary skill in the art. Thus, a diagnostic imaging amount of a complex or composition of the present invention to be administered may be selected in an amount sufficient such as to produce a diagnostic image of an organ or other site of the subject as described hereinabove. A therapeutically effective amount of a complex or composition of the present invention to be administered may be selected in an amount sufficient such as to produce a desired radiotherapeutic effect. More specifically a therapeutically effective amount is an amount of at least one of the complexes of the present invention which will permit sufficient tumor localization of the complex to stop and/or diminish tumor growth or size. As provided herein tumor growth or size can be monitored using the methods of the present invention or any other known diagnostic imaging procedure. Clearly the specific activity of the radionuclide of choice, e.g. ^{99m}Tc, ^{186/188}Re, ¹¹¹In⁺³, ^{67/68}Ga⁺³, ⁹⁰Y⁺³, ¹⁰⁹ Pd⁺², ¹⁰⁵Rh⁺³, ¹⁷⁷Lu, ^{64/67}Cu ¹⁶⁶Ho, ²¹³Bi, preferably Tc-99m, Re-186 or Re-188, will be taken into consideration in determining a dosage for diagnostic imaging or radiotherapy.

Generally, the unit dose to be administered has a radioactivity of about 0.01 mCi to about 300 mCi, preferably 10 mCi to about 200 mCi. For a solution to be injected a preferred unit dosage is from about 0.01 mL to about 10 mL. After e.g. intravenous administration, imaging of the organ or tumor in vivo can take place, if desired, from within minutes to hours or even longer, after the radiolabeled reagent has been administered to a subject. Typically, a sufficient amount of the administered dose will accumulate in the targeted area to be imaged within about 0.1 to 1 of an hour.

The complexes and/or compositions of the present invention may be administered by an appropriate route such as parentally (for example, intravenously), intramuscularly or intraperitoneally or by any other suitable method. For example, the complexes and/or compositions of this invention may be administered to a subject by bolus or slow infusion intravenous injection. The suitable forms for injection include sterile aqueous solutions or dispersions and sterile powders of the above mentioned complexes and/or compositions of the present invention.

The complexes or pharmaceutical compositions are preferably sterile. Sterilization can be accomplished by any art recognized technique, including but not limited to, addition of antibacterial of antifungal agents, for example, paraben, chlorobutanol, phenol, sorbic acid, thimerosal, and the like.

The complexes and/or compositions of the invention may also be used for in vitro detection of a cell expressing the folate receptor in a tissue biopsy taken from a subject. Thus in a further embodiment the present invention provides a method for in vitro detection of a cell expressing the folate receptor, e.g. a tumor cell, in a tissue sample which includes contacting said tissue sample with a complex or composition of the present invention in effective amounts and for sufficient time and conditions to allow binding to occur and detecting such binding by imaging techniques.

Samples can be collected by procedures known to the skilled person, e.g., by collecting a tissue biopsy or a body fluid, by aspirating for tracheal or pulmonary samples and the like.

Tissue samples to be tested include any tissue suspected to contain a cell expressing a folate receptor, such as tumor cells, epithelial cells, kidneys, gastrointestinal or the hepatobiliary system, and others. Samples can be sectioned, e.g., with a microtome, to facilitate microscopic examination and observation of bound complex. Samples can also be fixed with an appropriate fixative either before or after incubation with one of the complexes or compositions of the present invention to improve the histological quality of sample tissues.

Time and conditions sufficient for binding of a complex of the present invention to a folate receptor on the cell include standard tissue culture conditions, i.e. samples can be cultured in vitro and incubated with one of the complexes or compositions of the present invention in physiological media. Such conditions are well known to the skilled person. Alternatively, samples can be fixed and then incubated with a complex or composition of the present invention in an isotonic or physiological buffer.

A typical amount of said complex of the present invention for in vitro detection of a tumor cell can range from about 1 ng/l to about 1000 μg/l. A preferred amount is about 1 μg/l to about 100 μg/l. Preferred complexes to be used for in vitro diagnosis of a tumor cell are the same as for in vivo applications and include ^{99m}Tc, ^{186/188}Re, ¹¹¹In⁺³, ^{67/68}Ga⁺³, ⁹⁰Y⁺³, ¹⁰⁹Pd⁺², ¹⁰⁵Rh⁺³, ¹⁷⁷Lu, ^{64/67}Cu ¹⁶⁶Ho, ²¹³Bi, preferably Tc-99m, Re-186 or Re-188.

For detection of cellular binding of one of the present compounds, samples can be incubated in the presence of a selected complex, then washed and counted in a standard scintillation counter. Alternative methods apply and are known to the skilled person.

For diagnostic or radiotherapy applications it is convenient to prepare the complexes of the present invention at, or near, the site where they are to be used. Thus in a further aspect the present invention provides a single or multi-vial kit containing all of the components needed to prepare the complexes or compositions of this invention, other than the radionuclide ion itself. Thus a preferred single-vial kit of the present invention comprises a compound of the present invention, and a source of a pharmaceutically acceptable reducing agent such as a stannous salt. In addition, the kit comprises optionally further additives, for example the kit is buffered with a pharmaceutically acceptable acid or base to adjust the pH to a desired value for complex formation. Such a single vial kit may optionally contain exchange ligands such as glucoheptonate, gluconate, mannitol, maleate, citric or tartaric acid and may also contain reaction modifiers, such as diethylenetriaminepentaacetic acid or ethylenediamine tetraacetic acid. Additional additives, such as solubilizers (for example a cyclodextrin), antioxidants (for example ascorbic acid) and/or fillers (for example, NaCl) may be employed to improve the radiochemical purity and stability of the final product, or to aid in the production of the kit. The radionuclide, e.g. Tc or Re, will preferably be added separately in the form of a solution, e.g. a pertechnetate or perrhenate solution.

A preferred multi-vial kit of the present invention comprises, in one vial, the components, other than the radionuclide itself, required to form a labile radionuclide complex, that is, an exchange ligand and a pharmaceutically acceptable reducing agent such as a stannous salt. A compound of the present invention is contained in a second vial, as well as optional additives such as buffers appropriate to adjust the pH to its optimal value. Optionally the radionuclide will be provided in form of a solution, e.g. for example a pertechnetate or perrhenate solution, to be added.

All components of a kit may be in liquid, frozen or dry form. In a preferred embodiment, kit components are provided in lyophilized form.

All of the compounds, complexes, compositions and/or methods disclosed and claimed herein can be made and executed without undue experimentation in light of the present disclosure. It will be apparent to those of skill in the art that variations may be applied to the present invention without departing from the scope of the invention. The Examples provided herein are intended to be illustrative and are not exhaustive; therefore the illustrated Examples should not be viewed as limiting the invention in any way.

### EXAMPLES

### Materials and Methods

Melting points were taken on a Büchi-535 apparatus and are uncorrected. Infrared spectra were recorded on a Jasco FT/IR-6200 ATR-IR. Nuclear magnetic resonance spectra were recorded with a Bruker 300 MHz, 400 MHz or 500 MHz spectrometer with the corresponding solvent signals as an internal standard. Chemical shifts δ are reported in parts per million (ppm) relative to tetramethylsilane (0.00 ppm). Values of the coupling constant, ⁿJ, are given in Hertz (Hz); the following abbreviations are used in the experimental section for the description of ¹H-NMR spectra: singlet (s), broad singlet (bs), doublet (d), triplet (t), multiplet (m), doublet of doublets (dd). The chemical shifts of complex multiplets are given as the range of their occurrence. Low resolution mass spectra (LR-MS) were recorded with a Micromass Quattro Micro™ API LC-ESI and high resolution mass spectra (HR-MS) were recorded with a Bruker FTMS 4.7T BioAPEXII (ESI).

All water sensitive reactions were performed under argon in flame-dried glass ware. Reactions were monitored by thin layer chromatography (TLC, performed on EM Science 0.25 mm thick, precoated silica gel 60 F-254 glass supported plates) or HPLC. HPLC was performed on a Merck-Hitachi L-7000 system equipped with an L-7400 tunable absorption detector. The following systems were used for analytical HPLC: HPLC System 1: XBridge® column (C18, 5 μm, 4.6×150 mm, Waters); 0.1% TFA_{aq} (solvent A), acetonitrile (solvent B), 1 mL/min; 0-1 min, 95% A; 1-15 min, 95→5% A; 15-20 min, 5% A; 20→22 min, 5→95% A; 22→25 min, 95% A; HPLC System 2: XTerra® column (MSC18, 5 μm, 4.6×150 mm, Waters); 0-15 min 5→80% B; 15-20 min 95% B. Semiprep HPLC were performed with an XBridge column (C18, 5 μm, 10×150 mm, Waters) using the solvent system as indicated in the description of the individual experiments.

[Re(Br)₃(CO)₃][Et₄N]₂ was prepared according to Alberto et al, J. Chem. Soc. Dalton. Trans. 1994, 2815. [^{99m}Tc(H₂O)₃(CO)₃]⁺ was prepared according to Alberto et al, J. Am. Chem. Soc. 2001, 123, 3135.

[Na][^{99m}TcO₄]) was eluted from a ⁹⁹Mo/^{99m}Tc-generator (Mallinckrodt-Tyco, Petten, The Netherlands) with a 0.9% saline solution. The precursor [¹⁸⁸Tc(OH₂)₃(CO)₃]⁺ was synthesized using the Isolink™-kit (Mallinckrodt-Tyco, Petten, The Netherlands). For the in vitro studies and the biodistribution experiments, the radiofolates were separated from unlabeled folate ligand by means of HPLC in order to obtain a maximal specific activity. The non-purified complex used for SPECT/CT-studies.

### Example 1: Synthesis of Pte-Glu(H-His(τ-(4-N-Butyl))-OH)—OH (5-(4-(4-(2-amino-2-carboxyethyl)-1H-imidazol-1-yl)butylamino)-2-(4-((2-amino-4-oxo-3,4-dihydropteridin-6-yl)methylamino)benzamido)-5-oxopentanoic acid)

### (a) Synthesis of Boc-His (τ(4-NH₂Bu))-OMe

1-azido-4-chlorobutane has been prepared according to a modified based on Yao, L. et al, J. J. Org. Chem. 2004, 69, 1720. So 7.87 g (121 mmol) NaN₃ were suspended under argon in 220 ml dioxane. To the suspension 18.86 g of 1-bromo-4-chlorobutane were added and the mixture was stirred at room temperature for 18 hours. After addition of 550 ml water the mixture was extracted twice with 330 ml diethylether. The combined ether extracts were washed with 330 ml water and 330 ml aqueous sodium chloride (10%), dried over sodium sulphate and concentrated to give 14.11 g of a yellow pale oil with a purity of approximately 95% 1-azido-4-chlorobutane (¹H-NMR (300 MHz, CDCl₃): δ=1.65-1.95 (m, C(2)H₂, C(3)H₂, 4H); 3.33 (t, ³J=3.3, C(1)H₂, 2H); 3.57 (t, ³J=6.2, C(4)H₂, 2H).

To a suspension of 24, 24 g (90 mmol, 1.0 eq.) Boc-His-OMe (purchased from Bachem) in 50 ml acetone under argon was added 13.68 g (99 mmol, 1.1 eq.) potassium carbonate, 13.22 g (99 mmol, 1.1 eq.) 1-azido-4-chlorobutane and 3.75 g (25 mmol, 0.28 eq.) sodium iodide and the mixture was heated to reflux until TLC indicated a complete conversion (TLC: SiO₂, dichloromethane/methanol/9:1, Rf (product)=0.58, Rf (starting material)=0.37). After 2 days approximately 85% of starting material were consumed. The product was isolated by chromatography (SiO₂, ethylacetate/n-hexane/4:1) to give 11.74 g of Boc-His (τ(4-N₃Bu))-OMe as a yellow-brownish resin (¹H-NMR (300 MHz, CDCl₃): δ=1.44 (s, C(CH₃)₃, 9H); 1.5-1.6 (m, C_{Bu(3)}H₂, 2H); 1.8-1.9 (m, C_{Bu(2)}H₂, 2H); 3.00 (dd, ²J=14.4, ³J=4.7, C_{β-His}H_{A}, 1H); 3.08 (dd, ²J=14, 8; ³J=5.5, C_{β-His}H_{B}, 1H); 3.31 (t, ³J=6.6, N₃C_{Bu(4)}H₂, 2H); 3.70 (s, CH₃, 3H); 3.90 (t, ³J=7.0, C_{Bu(1)}H₂, 2H); 4.53 (ddd, C_{α-His}H, 1H); 5.99 (d, ³J=8.2, NH, 1H); 6.68 (s, Cᵢₘ₍₅₎H, 1H); 7.37 (s, Cᵢₘ₍₂₎H, 1H)).

To a solution of 11.73 g (32 mmol, 1.0 eq.) of the above obtained intermediate Boc-His (τ(4-N₃Bu))-OMe in 320 ml tetrahydrofuran under argon 10.49 g (40 mmol, 1.25 eq.) triphenylphosphine were added. The mixture was stirred at room temperature for 19 hours. After addition of 32 ml of water and further stirring for 4 hours the mixture was concentrated under vacuum to give 21.70 g of the desired heterocyclic ligand site Boc-His(τ(4-NH₂Bu))-OMe as a yellowish resin which contained approx. 50% triphenylphosphine oxide.

¹H-NMR (300 MHz, CDCl₃): δ=1.43 (s, C(CH₃)₃, 9H); 1.4-1.55 (m, C_{Bu(3)}H₂, 2H); 1.7-1.9 (m, C_{Bu(2)}H₂, 2H); 2.69 (t, ³J=6.9, C_{Bu(4)}H₂, 2H); 3.00 (dd, C_{β-His}H_{A}, 1H); 3.08 (dd, C_{β-His}H_{B}, 1H); 3.70 (s, CH₃, 3H); 3.88 (t, ³J=7.0, C_{Bu(1)}H₂, 2H); 4.52 (ddd, C_{α-His}H, 1H); 5.95 (d, ³J=7.9, NH, 1H); 6.67 (s, Cᵢₘ₍₅₎H, 1H); between 7.20 and 7.75 (s, Cᵢₘ₍₂₎H, 1H).

Triphenylphosphine oxide: δ=7.20-7.75 (m, Ph₃).

### (b) Synthesis of H-Glu(NH(Boc-His(τ-Bu-4-yl)OMe)-OtBu

To a solution of 4.26 g (10 mmol, 1.0 eq.) Fmoc-Glu-OtBu (purchased from Bachem) in 40 ml monoglyme (1,2-dimethoxyethane) 1.38 g (12 mmol, 1.2 eq.) of N-hydroxy-succinimide were added. The mixture was cooled to 5° C. and 2.48 g (12 mmol, 1.2 eq.) of N,N′-dicyclohexyl-carbodiimide (DCC) were added. After stirring for 22 hours at room temperature all starting material was consumed (TLC, SiO₂, ethylacetate/hexane/1:1, Rf_{product}=0.43, Rf_{Starting material}=0.35) and a solution was formed which was directly used in the following steps.

Thus, to the above obtained solution of Fmoc-Glu (OSu)-OtBu in 40 ml monoglyme (1,2-dimethoxyethane), 6.88 g (10 mmol, purity ˜50%, 1.0 eq.) of Boc-His (τ(4-NH₂Bu))-OMe (see step (a)) were added. After two hours stirring at ambient temperature the mixture was concentrated to approximately 15 g and the product was purified by flash-chromatography (SiO₂, dichloromethane/methanol/25:1, TLC, SiO₂, dichloromethane/methanol 9:1, Rf_{product}=0.46, Rf_{starting material}=0.05) to give 2.29 g of Fmoc-Glu (NH(Boc-His (τ-Bu-4-yl)OMe)-OtBu as a yellowish foam (¹H-NMR (300 MHz, CDCl₃): δ=1.39 (s, C(CH₃)₃, 9H); 1.3-1.5 (m, C_{Bu(3)}H₂, 2H); 1.44 (s, C(CH₃)₃, 9H); 1.63-1.75 (m, C_{Bu(2)}H₂, 2H); 1.85-1.95 (m, C_{β-Glu}H_{A}, 1H); 2.1-2.3 (m, C_{β-Glu}H_{B}, 1H); 2.24 (m, C_{γ-Glu}H₂, 2H); 2.97 (2 dd, C_{β-His}H₂, 2H); 3.1-3.3 (m, C_{Bu(4)}H₂, 2H); 3.64 (s, CH₃, 3H); 3.79 (t, C_{Bu(1)}H₂, 2H); 4.1-4.25 (t, C_{α-Glu}H; t, C_{β-Fmoc}H, 2H); 4.25-4.45 (m, C_{α-Fmoc}H₂, 2H); 4.52 (q, C_{α-His}H, 1H); 5.82 (d, ³J=7, 6, N_{α-Glu}H, 1H); 5.95 (d, ³J=8.1, N_{α-His}H, 1H); 6.36 (s, N_{γ-Glu}H, 1H); 6.61 (s, Cᵢₘ₍₅₎H, 1H); 7.20-7.30 (s, Cᵢₘ₍₂₎H, 1H); between 7.20 and 7.80 (m, C_{Fmoc}H₈, 8H))).

To a mixture of 2.29 g (3.06 mmol, 1.0 eq.) Fmoc-Glu(NH(Boc-His(t-Bu-4-yl)OMe)-OtBu and 50 ml dichlormethane 3.0 ml piperidine were added. After 3 hours stirring at room temperature the product was isolated by flash chromatography (SiO₂, dichloromethane/methanol 4:1, TLC, SiO₂, dichloromethane/methanol 9:1, Rf_{product}=0.20, Rf_{starting material}=0.51) to give 892 mg of H-Glu(NH(Boc-His(τ-Bu-4-yl)OMe)-OtBu as a pink foam.

¹H-NMR (300 MHz, CDCl₃): δ=1.42 (s, C(CH₃)₃); 1.35-1.55 (m, CBu(₃)H₂); 1.46 (s, (CH₃)₃); 1.75-1.9 (m, C_{Bu(2)}H₂; m, C_{b-Glu}H_{A}); 2.05-2.2 (m, C_{β-Glu}H_{B}); 2.35 (“t”, C_{γ-Glu}H₂); 2.95-3.1 (2 dd, C_{β-His}H₂); 3.22 (q, C_{Bu(4)}H₂); 3.41 “dd”, C_{α-Glu}H); 3.67 (s, CH₃); 3.90 (t, C_{Bu(1)}H₂); 4.53 (ddd, C_{α-His}H); 5.94 (d, ³J=8, 4, N_{α-His}H); 6.65-6.7 (s, NH; s, Cᵢₘ₍₅₎H); 7.42 (s, Cᵢₘ₍₂₎H)

### (c) Synthesis of Pte-Glu(H-His(τ-(4-N-Butyl))-OH)—OH (5-(4-(4-(2-amino-2-carboxyethyl)-1H-imidazol-1-yl)butylamino)-2-(4-((2-amino-4-oxo-3,4-dihydropteridin-6-yl)methylamino)benzamido)-5-oxopentanoic acid)

A mixture of 25.3 g pteroic acid and 1175 ml of formic acid was refluxed for 3 hours. After cooling to room temperature 2350 ml of methyl-tert.butyl ether were added. The resulting suspension was stirred for 2 hours at room temperature, the precipitate was filtered off, washed with 1600 ml of water and dried under vacuum at 40° C. to give 27.28 g of 10-formylpteroic acid (¹H-NMR (300 MHz, DMSO-d₆): δ=12.90 (bs, COOH, 1H); 11.40 (bs, N(3)H, ¹H); 8.85 (s, CHO, 1H); 8.65 (s, C(7)H, 1H); 7.95 (d, Ph, 2H); 7.56 (d, Ph, 2H); 6.90 (bs, NH₂, 2H)).

To a suspension of 8.5 g of the above obtained 10-formylpteroic acid in 128 ml N,N-dimethylformamide, 52 ml N,N-dimethylformamide-diisopropylacetal were added. The mixture was stirred at room temperature for 18 hours. The precipitate which has formed was filtered off, washed with 4 ml of N,N-dimethylformamide and 50 ml acetone and dried at 40° C. under vacuum to give 8.7 g of protected N²,N,N-dimethylaminomethylene-10-formylpteroic acid (¹H-NMR (300 MHz, DMSO-d₆+D₂O): δ=8.70 (s, CHN, 1H); 8.63 (s, C(7)-H, CHO, 2H); 7.81 (d, Ph, 2H); 7.30 (d, Ph, 2H); 5.18 (C(6)CH₂, 2H); 3.19 (s, NCH₃, 3H); 3.08 (s, NCH₃, 3H)).

To a solution of 391 mg (0.90 mmol, 1.0 eq.) of the above obtained N²,N,N-dimethylaminomethylene-10-formyl-pteroic acid in 3 ml tetrahydrofuran under argon were added 274 mg (0.99 mmol, 1.1 eq.) DMTMM (4-(4,6-Dimethoxy[1,3,5]triazin-2-yl)-4-methylmorpholiniumchlorid, synthesized according to Kunishima et al, Tetrahedron Letters, 40, 5327-5330, 1999) and 473 mg (0.99 mmol, 1.1 eq.) H-Glu(NH(Boc-His(t-Bu-4-yl)OMe)-OtBu (see step (b)). The suspension was stirred for three hours at room temperature and then concentrated under vacuum to approximately 1.5 g. After addition of 2 ml water a precipitate formed which was separated from the solution by centrifugation. The precipitate was resuspended in 0.5 ml water, separated by centrifugation and dried under vacuum to give 526 mg of N²-N,N-dimethylaminomethylene-10-formyl-Pte-Glu(NH(Boc-His(τ-Bu-4-yl)OMe)-OtBu as a yellow foam.

¹H-NMR (300 MHz, CDCl₃): δ=1.39 (s, C(CH₃)₃); 1.3-1.45 (m, C_{Bu(3)}H₂); 1.46 (s, C(CH₃)₃); ˜1.7-1.8 (m, C_{Bu(2)}H₂); 1.8-1.9 (m, C_{β-Glu}H_{A}); 2.1-2.4 (m, C_{β-Glu}H_{B}; m, C_{γ-Glu}H₂); 2.95-3.05 (2 dd, C_{β-His}H₂); 3.14 (s, C_{DMAM}H_{3A}); 3.22 (s, C_{DMAM}H_{3B}); 3.2-3.3 (m, C_{Bu(4)}H₂); 3.69 (s, CH₃); 3.8-3.9 (m, C_{Bu(1)}H₂); 4.4-4.6 (m, C_{α-His}H; m, C_{α-Glu}H); 5.29 (s, C_{Pte(9)}H₂); 5.60 (d, NH); 5.94 (d, NH); 6.70 (s, Cᵢₘ₍₅₎H); 6.95 (s, NH); 7.40 (d, ³J=8.6, 2×C_{Pte(5′)}H); 7.48 (s, Cᵢₘ₍₂₎H); 7.87 (d, ³J=8.6, 2×C_{Pte(6′)}H); 8.70 (s, C_{DMAM}H); 8.80 (s, C_{For}H); 8.93 (s, C_{Pte(7)}H).

To 200 mg (0.22 mmol, 1 eq.) of the above obtained N²-N,N-dimethylaminomethylene-10-formyl-Pte-Glu(NH(Boc-His(t-Bu-4-yl)OMe)-OtBu 22 ml 1M HCl were added. The mixture was stirred for 2 hours at 50° C. After cooling to approximately 15° C. 1.76 g solid sodium hydroxide were added. After stirring at room temperature for one hour the pH was adjusted to pH=2.5 by addition of formic acid. The product was isolated by reversed phase medium pressure liquid chromatography (RP-MPLC, solid phase: Europrep 60-60 C-18; 60 Å; 35-70 μm, 140 g; 36 cm×26 mm, liquid phase: 0-10 min. 99.9% H₂O, 0.1% HCOOH, 10-40 min. 34.9% MeOH, 65% H₂O, 0.1% HCOOH) to give 120 mg of Pte-Glu(H-His(τ-(4-N-Butyl))-OH)—OH as a yellowish resin.

¹H-NMR (300 MHz, D₂O&D₂SO₄, cal.: δ(H₂O)=4.79): δ=0.4-0.6 (m, C_{Bu(3)}H₂); 0.75-0.9 (m, C_{Bu(2)}H₂); ˜1.1-1.25 (m, C_{β-Glu}H_{A}); 1.25-1.4 (m, C_{β-Glu}H_{B}); 1.50 (t, ³J=7.1, C_{γ-Glu}H₂); 2.1-2.3 (m, C_{Bu(4)}H₂); 2.45 (2 dd, C_{β-His}H₂); 3.1-3.3 (t, C_{Bu(1)}H₂); 3.4-3.5 (t, C_{α-His}H); 3.5-3.6 (q, ³J_{E}=4.6, ³J_{Z}=9.44, C_{α-Glu}H); 4.08 (s, C_{Pte(9)}H₂); ˜6.49 (s, Cᵢₘ₍₅₎H); 6.65 (d, ³J=6.7, 2×C_{Pte(5′)}H); 6.95 (d, ³J=6.1, 2×C_{Pte(6′)}H); 7.68 (s, Cᵢₘ₍₂₎H); 7.83 (s, C_{Pte(7)}H).

### Example 2: Synthesis of Re(CO)₃—His-folate Complex

Pte-Glu(H-His(τ-(4-N-Butyl))-OH)—OH (5-(4-(4-(2-amino-2-carboxyethyl)-1H-imidazol-1-yl)butylamino)-2-(4-((2-amino-4-oxo-3,4-dihydropteridin-6-yl)methylamino)benzamido)-5-oxopentanoic acid) (15.0 mg, 23 μmol) obtained according to Example 1 and [Re(Br)₃(CO)₃][Et₄N]₂ (20.0 mg, 26 μmol) were suspended in H₂O/MeOH (4 mL, 1:1) and the pH was adjusted to pH 8 with dilute NaHCO₃. The resulting yellow solution was stirred at 50° C. for 1.5 h after which HPLC indicated complete conversion of the starting material. The mixture was cooled to rt and the pH adjusted to pH 2-3 by addition of dilute HCl (0.1 M). The precipitate was isolated by centrifugation (10 min, 3500 rpm) and dried under reduced pressure to provide Re-complex (5) as a brown solid: HR-MS: [M+H]⁺=920.2131 (calc. for C₃₂H₃₅N₁₁O₁₀Re: 920.2126), HPLC purity: >70%.

### Example 3: Synthesis of ^{99m}Tc(CO)₃His-folate Complex

In analogy to Example 2, ^{99m}Tc(CO)₃-His-folate was prepared by addition of a stock solution of the Pte-Glu(H-His(τ-(4-N-Butyl))OH)—OH (5-(4-(4-(2-amino-2-carboxyethyl)-1H-imidazol-1-yl)butylamino)-2-(4-((2-amino-4-oxo-3,4-dihydropteridin-6-yl)methylamino)benzamido)-5-oxopentanoic acid) obtained according to Example 1 in phosphate buffered saline (PBS) to [Na][^{99m}TcO₄]) resulting in a final concentration of 10⁻⁵ M.

The sealed reaction vial was heated for 60 min at 100° C. to form the corresponding in excellent yield (>98%).

### Example 4: Synthesis of Triazol-Folate

### a) Synthesis of 4-azido-butane-amine

The Boc-protected intermediate azide (0.42 g, 2.0 mmol; prepared according to Link et al J. Am. Chem. Soc. 2004, 126, 10598) was dissolved in CH₂Cl₂ (5 mL) and trifluoroacetic acid (TFA, 1.0 mL) was added. The mixture was left at rt over night and then concentrated under reduced pressure to yield the TFA salt of amine the corresponding Azide-Amine as a colorless oil (450 mg, quantitative): ¹H-NMR (CDCl₃) δ=8.19-7.80 (bs, 3H), 4.76-4.50 (bs, 1H), 3.32 (t, 2H, J=6.5), 3.30-2.92 (m, 2H), 1.81-1.70 (m, 2H), 1.70-1.58 (m, 2H); LR-MS: [M+H]⁺=115.10 (calc. for C₄H₁₀N₄: 114.15).

### b) Synthesis of Glu(4-azido-butylamide)OMe

In a flamed-dried flask under argon was dissolved BocGluOMe (261 mg, 1.0 mmol) in dry DMF (5 mL, over molecular sieves 4 Å) and Et₃N (210 μL, 1.5 equiv) was added. HBTU (380 mg, 1.0 mmol) was added at 0° C. and the mixture was stirred for half an hour. The solution of the activated acid was transferred via cannula to a solution of the TFA salt of the azide-amine obtained under a) (228 mg, 1.0 mmol) in dry DMF (5 mL) containing Et₃N (210 μL, 1.5 equiv) at 0° C. After 2 hrs, the mixture was warmed to rt and stirred over night. Removal of volatile components under reduced pressure and purification of the residue by flash chromatography on silicagel with CH₂Cl₂/MeOH (60:1→30:1) provided the corresponding Boc-protected amide product as a colorless oil (330 mg, 92%): ¹H-NMR (CDCl₃) δ=6.32-6.19 (bs, 1H), 5.35-5.24 (bs, 1H), 4.30-4.21 (m, 1H), 3.72 (s, 3H), 3.35-3.21 (m, 4H), 2.24 (t, 2H, J=6.8), 2.21-2.10 (m, 1H), 195-1.80 (m, 1H), 1.67-1.52 (m, 4H), 1.43 (s, 9H); LR-MS: [M+H]⁺=358.20 (calc. for C₁₅H₂₇N₅O₅: 357.41).

The Boc-protected intermediate obtained above (0.72 g, 2.0 mmol) was dissolved in CH₂Cl₂ (10 mL) and trifluoroacetic acid (TFA, 1.5 mL) was added. The mixture was left at rt over night and then concentrated under reduced pressure to yield the TFA salt of the corresponding amine as a pale yellow oil (740 mg, quantitative): ¹H-NMR (CDCl₃) δ=10.15-8.60 (bs, 3H), 6.74 (t, 1H, J=5.6), 4.14 (dd, 1H, J=7.7 and 3.7), 3.80 (s, 3H), 3.31-3.26 (m, 2H), 3.25-3.18 (m, 2H), 2.59-2.45 (m, 2H), 2.38-2.27 (m, 1H), 2.24-2.13 (m, 1H), 1.63-1.50 (m, 4H); LR-MS: [M+H]⁺=258.23 (calc. for C₁₀H₁₉N₅O₃: 257.29).

### c) Synthesis of protected γ-(4-azido-butaonyl)-folic acid amide

In a flamed-dried flask under argon was suspended N2-N,N-dimethylaminomethylene-10-formyl-pteroic acid (198 mg, 0.5 mmol) in dry DMF (10 mL, over molecular sieves 4 Å) and Et₃N (104 μL, 0.75 mmol) was added. HBTU (380 mg, 0.5 mmol) was added at 0° C. and the mixture was stirred for one hour. To the resulting orange solution was added at 0° C. a solution of amine TFA salt obtained under c) (186 mg, 0.5 mmol) in dry DMF (9 mL) containing Et₃N (210 μL, 1.5 mmol). The resulting clear yellow solution was stirred at 0° C. for one hour and then allowed to warm to rt. Removal of volatile components under reduced pressure and purification of the residue by flash chromatography on silicagel with CH₂Cl₂/MeOH (17:1→10:1) provided the corresponding protected azido-folate as a yellow solid (290 mg, 92%): mp 125-130° C.; HR-MS: [M+Na]⁺=657.2617 (calc. for C₉H₁₅N₄O₄Na: 657.2624).

The under d) obtained product (63 mg, 0.1 mmol) was dissolved in 1 M NaOH (3 mL) and stirred at rt over night. The resulting turbid solution was cleared by filtration through Celite™. The pH of the yellow solution was adjusted to pH˜2 by addition of HCl (first 37% HCl, then 1 M HCl) which resulted in precipitation of the product. The suspension was centrifuged (10 min at 3500 rpm), the pale yellow supernatant decanted and the solid product dried under reduced pressure to give the penta-hydrochloride salt of azido folate as a yellow powder (75 mg, quantitative): mp>200° C.; ¹H-NMR (DMSO-d6) δδ=12.21-11.95 (bs, 1H), 8.64 (s, 1H), 8.18 (d, 1H, J=7.2), 7.85 (t, 1H, J=5.7), 7.65 (d, 2H, J=9.0), 7.00-6.82 (bs, 2H), 6.93 (t, 1H, J=6.2), 6.64 (d, 2H, J=9.0), 4.49 (d, 2H, J=5.9), 4.32-4.22 (m, 1H), 3.29 (t, 2H, J=6.8), 3.03 (q, 2H, J=6.5), 3.09-2.96 (m, 2H), 2.12-1.83 (m, 2H), 1.55-1.45 (m, 2H), 1.45-1.35 (m, 2H) (one NH not observed); HR-MS: [M]′=537.2127 (calc. for C₂₃H₂₇N₁₁O₅: 537.2197); elemental analysis (calculated %-values for C₂₃H₂₇N₁₁O₅(HCl)₅ in parenthesis) C, 39.16 (38.38); H, 4.09 (4.48); N, 21.43 (21.40); O, (11.11); Cl, (24.63).

### f) Synthesis of Triazol-folate

Synthesis A:

Protected azido folate (95 mg, 0.15 mmol, obtained under d)) was dissolved in ^{t}BuOH/H₂O (1:1, 6 mL) and L-propargyl glycine (17 mg, 0.15 mmol), Cu(OAc)₂ (5.5 mg, 20 mol %) and sodium ascorbate (12 mg, 40 mol %) were added. The brown solution was stirred at rt over night after which HPLC indicated completed conversion of the substrates (HPLC system 1). Metal scavenger resin QuadraPure IDA™ (0.3 g) was added and the mixture was kept at rt for one day while occasionally shaken. The resulting yellow solution was decanted and concentrated under reduced pressure. The residue was taken up in 1 M NaOH (4 mL) and stirred at rt over night after which HPLC indicated complete deprotection of intermediate X. The pH of the yellow solution was adjusted to pH ˜2 by addition of HCl (first 37% HCl, then 1 M HCl) which resulted in precipitation of the product. The suspension was centrifuged (10 min at 3500 rpm), the pale yellow supernatant decanted and the solid product dried under reduced pressure. HPLC purification of the crude product (XBridge semiprep column, isochratic 10% CH₃CN, 90% aq. TFA (0.1%)) provided the tris-TFA salt, mono-hydrate of the desired Triazol-folate as a yellow powder (113 mg, 75% for 2 steps): ¹H-NMR (DMSO-d6, using a water-signal suppressing program) δ=8.68 (bs, 1H), 8.27 (bs, 2H, exchanged with D₂O), 8.20 (d, 1H, exchanged with D₂O, J=6.8), 7.92 (s, 1H), 7.85 (t, 1H, exchanged with D₂O, J=5.7), 7.65 (d, 2H, J=8.1), 7.5-7.0 (multiple bs, 2H, exchanged with D₂O), 6.64 (d, 2H, J=8.1), 4.50 (s, 2H), 4.35-4.20 (m, 5H, one proton exchanges with D₂O), 3.23-3.13 (m, 2H), 3.09-2.94 (m, 2H), 2.18 (t, 2H, J=8.2), 2.10-1.98 (m, 1H), 1.93-84 (m, 1H), 1.80-1.72 (m, 2H), 1.45-1.27 (m, 2H); HR-MS: [M+H]⁺=651.2738 (calc. for C₂₈H₃₄N₁₂O₇: 650.2673); elemental analysis (calculated %-values for C₂₈H₃₄N₁₂O₇(TFA)₃ (H₂O) in parenthesis) C, 40.30 (40.40); H, 4.20 (3.98); N, 16.86 (16.63); O (22.16); content of fluoride determined by titration: 14.95 (calc.: 16.63).

Synthesis B:

Deprotected azido folate (36 mg, 0.05 mmol; obtained under e)) was suspended in ^{t}BuOH/H₂O (1:1, 3 mL) and L-propargyl glycine (6 mg, 0.053 mmol), Cu(OAc)₂ (2 mg, 20 mol %) and sodium ascorbate (4 mg, 40 mol %) were added. The mixture was stirred at 80° C. for 20 min after which HPLC (HPLC system 1) indicated completed conversion of the starting substrate. The brown suspension was dissolved by addition of 1M NaOH and as cleared by filtration through Celite™. The product was precipitated by adjusting the pH of the solution to pH˜2 with 1 M HCl. The suspension was centrifuged (10 min at 3500 rpm), the supernatant decanted and the solid product dried under reduced pressure yielding the penta-hydrochloride salt of the desired Triazol-folate as an orange solid (42 mg, quantitative).

### Example 5: Synthesis of ^{99m}Tc(CO)₃-Triazol-folate Complex

Synthesis A:

50 μL of a stock solution (10² M to 10⁻² M in physiological (0.15 M) phosphate buffer pH=7.4) of the Triazol-folate ligand was added to a solution of [^{99m}Tc(CO)₃(OH₂)₃]⁺ (prepared according to Alberto et al, J. Am. Chem. Soc. 2001, 123, 3135; 100 μL; ˜1 GBq/mL). Phosphate buffered saline (PBS pH 7.4, 350 μL) was added to adjust the final concentration. The reaction was heated for 50 min at 100° C. Radiolabeling yields were determined via HPLC. Complexes were analyzed via HPLC and the identity confirmed according to common practice by comparison with the UV trace of the corresponding Re-complexes (see Example 6) using HPLC system 2.

One-Pot-Synthesis B:

Deprotected Azido folate (obtained under step 4e); 40 μL, ca. 10⁻³ M in MeOH/PBS pH 7.4 (5:1)) was mixed with L-propargyl glycine (20 μL, 10⁻² M in water), Cu(OAc)₂ (5 μL, 10⁻² M in water) and sodium ascorbate (20 μL, 10⁻² M in water). After heating to 100° C. for 30 min, the mixture was cooled to rt and added to [^{99m}Tc(H₂O)₃(CO)₃]⁺ (100 μL, ˜1 GBq/mL) in PBS (0.6 mL, 0.15 M, pH 7.4). After additional heating to 100° C. for 60 min, clean formation of the desired complex was confirmed by HPLC (HPLC system 2).

### Example 6: Synthesis of Re(CO)₃-Triazol-folate Complex

Triazol-folate (obtained hereinabove); tri-trifluoroactetate mono-hydrate, 0.7 mg, 0.7 μmol) and [Re(Br)₃(CO)₃][Et₄N]₂ (1.0 mg, 1.4 μmol) were suspended in water (0.5 mL). Addition of NaOH (0.1 M) to a final pH of 8 yielded a yellow solution which was stirred at 50° C. for 1 h after which HPLC (HPLC system 1) indicated complete conversion of the starting azido folate. The solution was cooled to rt and the pH adjusted to pH˜2 by addition of dilute aq. HCl. The precipitate was isolated by centrifugation (3 min 16,000 rpm), dissolved in NaOH (0.1 M) and purified by HPLC (HPLC system 1) to provide a reference solution of cold Re-complex in CH₃CN/0.1% TFA. HR-MS: [M+H]⁺=921.2074 (calc. for C₃₁H₃₄N₁₂O₁₀Re: 921.2078). HPLC purity >95%.

### Example 7: In Vivo Experiments

Biodistribution studies were performed with 4-5-week-old male, athymic nude mice (NMRI nu/nu; Charles River, The Netherlands). The animals were acclimated and fed with a folate-deficient rodent diet starting 5 days prior to the tumor cell inoculation. The mice were inoculated subcutaneously with the KB-tumor cell suspension (5×10⁶ cells) into the subcutis of each shoulder. Radiofolate biodistribution studies were carried out approx. 14 days after tumor cell inoculation when the tumor size reached a size of approx. 0.5-1.5 cm³. The experiments were performed in triplicate. The ^{99m}Tc(CO)₃-His-folate and ^{99m}Tc(CO)₃-Triazol-folate, respectively, (1.5 MBq in 100 μL) were administered via a lateral tail vein. For the experiments in combination with the antifolate, pemetrexed (PMX; Alimta®; Lilly, Bad Homburg, Germany) was diluted with NaCl 0.9% according to the instruction of the manufacturer. It was administered (400 μg in 100 μL) 1 h previous to the radiotracer via a lateral tail vein. The animals were sacrificed at 1 h, 4 h and 24 h after administration of ^{99m}Tc-radiofolates alone or with pre-injected PMX. The selected tissues were removed, weighed, and counted for radioactivity in a γ-counter to determine the percentage of injected activity per gram of tissue (% IA/g).

The biodistribution data obtained for ^{99m}Tc-His-folate (Tables 1a,b) and of ^{99m}Tc-Triazol-folate (Tables 2a,b) with or without preinjection with the antifolate Pe-metrexed (Tables 1b, 2b) in KB-tumor bearing male nude mice are shown in Tables 1 to 4. The values are indicated as percentage injected activity per gram tissue [% IA/g]. The results in Tables 1a and 2a clearly show that the compounds of the present invention achieve an excellent tumor-blood value.

The experiments in combination with the antifolate, pemetrexed, are shown in Tables 1b and 2b. The results indicate that with prior administration of an antifolate even higher specificity can be observed.

TABLE 1a

| | Time p.i. | | |
|---|---|---|---|
| Site | 1 h p.i. | 4 h p.i. | 24 h p.i. |
| blood | 0.18 ± 0.10 | 0.12 ± 0.02 | 0.02 ± 0.01 |
| heart | 3.60 ± 0.53 | 1.16 ± 0.36 | 0.14 ± 0.07 |
| lung | 1.04 ± 0.12 | 0.69 ± 0.09 | 0.16 ± 0.07 |
| spleen | 0.37 ± 0.22 | 0.30 ± 0.04 | 0.05 ± 0.01 |
| kidneys | 23.96 ± 10.11 | 24.56 ± 3.17 | 6.70 ± 1.12 |
| stomach | 1.71 ± 0.12 | 1.03 ± 0.22 | 0.15 ± 0.02 |
| intestines | 3.84 ± 2.23 | 1.51 ± 0.29 | 0.18 ± 0.14 |
| liver | 9.73 ± 1.32 | 3.83 ± 1.49 | 0.44 ± 0.31 |
| muscle | 1.56 ± 0.07 | 1.09 ± 0.26 | 0.19 ± 0.03 |
| bone | 0.77 ± 0.07 | 0.58 ± 0.20 | 0.06 ± 0.01 |
| parotid gland | 6.52 ± 1.04 | 5.72 ± 0.63 | 1.58 ± 0.69 |
| tumor right | 2.75 ± 0.62 | 4.35 ± 0.71 | 3.34 ± 0.34 |
| tumor left | 2.52 ± 0.40 | 4.23 ± 0.78 | 3.68 ± 0.36 |
| tumor-to-blood | 18.56 ± 10.47 | 38.00 ± 8.33 | 184.23 ± 65.88 |
| tumor-to-liver | 0.30 ± 0.06 | 1.19 ± 0.47 | 10.99 ± 5.60 |
| tumor-to-kidney | 0.12 ± 0.05 | 0.18 ± 0.03 | 0.53 ± 0.10 |

TABLE 1b

| | Pemetrexed* (Alimta ®); Time p.i. | | |
|---|---|---|---|
| | 1 h p.i. | 4 h p.i. | 24 h p.i. |
| blood | 0.09 ± 0.04 | 0.04 ± 0.01 | 0.01 ± 0.00 |
| heart | 1.57 ± 0.59 | 0.23 ± 0.04 | 0.08 ± 0.02 |
| lung | 0.72 ± 0.06 | 0.29 ± 0.08 | 0.07 ± 0.01 |
| spleen | 0.18 ± 0.09 | 0.08 ± 0.02 | 0.03 ± 0.01 |
| kidneys | 7.94 ± 2.27 | 3.40 ± 1.14 | 1.81 ± 0.48 |
| stomach | 1.58 ± 0.85 | 0.54 ± 0.11 | 0.43 ± 0.40 |
| intestines | 4.81 ± 4.65 | 0.50 ± 0.13 | 0.23 ± 0.24 |
| liver | 3.91 ± 2.24 | 1.24 ± 0.49 | 0.24 ± 0.06 |
| muscle | 1.02 ± 0.26 | 0.43 ± 0.08 | 0.18 ± 0.12 |
| bone | 0.53 ± 0.16 | 0.15 ± 0.02 | 0.04 ± 0.02 |
| parotid gland | 3.98 ± 1.17 | 1.68 ± 0.62 | 0.88 ± 0.23 |
| tumor right | 2.59 ± 0.62 | 3.56 ± 0.88 | 1.98 ± 0.24 |
| tumor left | 2.64 ± 0.71 | 4.21 ± 1.11 | 2.31 ± 0.62 |
| tumor-to-blood | 29.19 ± 4.82 | 110.10 ± 2.49 | 167.13 ± 56.47 |
| tumor-to-liver | 0.74 ± 0.18 | 3.05 ± 2.26 | 9.85 ± 4.63 |
| tumor-to-kidney | 0.34 ± 0.06 | 1.20 ± 0.37 | 1.28 ± 0.55 |

TABLE 2a

| | Time p.i. | | | |
|---|---|---|---|---|
| | | | | excess |
| | | | | folic acid |
| Site | 1 h p.i. | 4 h p.i. | 24 h p.i. | 4 h p.i. |
| blood | 0.12 ± 0.03 | 0.12 ± 0.04 | 0.02 ± 0.01 | 0.22 ± 0.36 |
| heart | 2.17 ± 0.45 | 0.83 ± 0.05 | 0.11 ± 0.02 | 0.02 ± 0.02 |
| lung | 0.91 ± 0.23 | 0.63 ± 0.08 | 0.11 ± 0.07 | 0.05 ± 0.07 |
| spleen | 0.38 ± 0.14 | 0.32 ± 0.07 | 0.06 ± 0.03 | 0.02 ± 0.04 |
| kidneys | 18.11 ± 2.53 | 27.33 ± 3.61 | 8.03 ± 3.40 | 0.18 ± 0.09 |
| stomach | 1.39 ± 0.18 | 1.02 ± 0.04 | 0.15 ± 0.03 | 0.07 ± 0.05 |
| intestines | 2.47 ± 0.94 | 0.76 ± 0.14 | 0.28 ± 0.27 | 1.26 ± 0.65 |
| liver | 2.95 ± 1.02 | 0.89 ± 0.42 | 0.14 ± 0.02 | 1.48 ± 1.26 |
| muscle | 1.55 ± 0.22 | 0.82 ± 0.16 | 0.16 ± 0.08 | <0.01 |
| bone | 0.81 ± 0.17 | 0.48 ± 0.11 | 0.05 ± 0.02 | <0.01 |
| parotid gland | — | — | — | — |
| tumor right | 3.76 ± 1.03 | 5.35 ± 0.33 | 2.98 ± 0.76 | 0.09 ± 0.07 |
| tumor left | 4.61 ± 1.00 | 4.33 ± 1.00 | 3.37 ± 2.06 | 0.02 ± 0.03 |
| tumor-to-blood | 38.37 ± 16.16 | 42.14 ± 12.09 | 138.12 ± 40.31 | |
| tumor-to-liver | 1.58 ± 0.62 | 6.29 ± 2.74 | 22.28 ± 8.00 | |
| tumor-to-kidney | 0.24 ± 0.09 | 0.18 ± 0.04 | 0.39 ± 0.09 | |

TABLE 2b

| | Pemetrexed* (Alimta ®); Time p.i. |
|---|---|
| Site | 4 h p.i. |
| blood | 0.05 ± 0.04 |
| heart | 0.34 ± 0.15 |
| lung | 0.31 ± 0.15 |
| spleen | 0.08 ± 0.03 |
| kidneys | 4.22 ± 2.28 |
| stomach | 0.70 ± 0.26 |
| intestines | 0.53 ± 0.30 |
| liver | 0.49 ± 0.30 |
| muscle | 0.35 ± 0.08 |
| bone | 0.18 ± 0.04 |
| parotid gland | — |
| tumor right | 2.79 ± 0.67 |
| tumor left | 2.56 ± 0.42 |
| tumor-to-blood | 68.31 ± 33.15 |
| tumor-to-liver | 7.16 ± 4.49 |
| tumor-to-kidney | 0.71 ± 0.33 |

*400 μg, injected 1 h previous to the radiotracer

### Example 8: Ex Vivo/In Vitro Autoradiography

Ex Vivo Autoradiography:

Immediately after euthanasia, tumors and kidneys were removed and frozen, embedded in TissueTek at −80° C. Frozen tumors and kidneys were cut into sections of 10 μm with a microtom (Cryo-Star HM 560 M, Walldorf, Germany) and mounted on slides (Superfrost plus, Menzel, Braunschweig, Germany). The slides were exposed to phosphor imaging screens (Multisensitive screen, Packard Instruments Co., Meriden, USA) in X-ray cassettes over night. The screens were then read by a phosphor imager (Cyclone, Packard, Instruments Co., Groningen, The Netherlands).

In Vitro Autoradiography:

In vitro autoradiography was performed on adjacent sections of those prepared from tumor and kidneys for ex vivo autoradiography. The slides with tumor sections were pre-incubated in Tris-HCL buffer 8170 mM, pH 7.6, with 5 mM MgCl₂) with 0.25 (w/v) BSA for 10 min at room temperature. Then, the sections were incubated with a solution of ^{99m}Tc-His-folate or ^{99m}Tc-Triazol-folate (0.5 MBq/mL in Tris-HCl buffer, containing 1% BSA) for 60 min at RT. After incubation, the sections were rinsed twice for 5 min in cold Tris-HCl buffer (with 25% BSA), then washed for 5 min in pure Tris-HCl buffer and finally rinsed with cold MilliQ. The sections were air-dried, exposed to phosphor imaging screens.

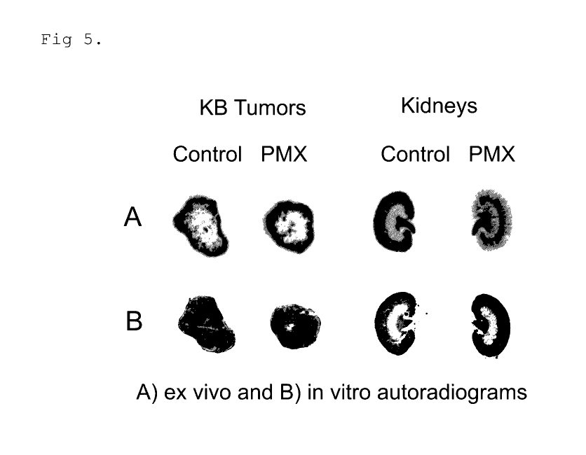

The results are shown in FIG. 5.

### Example 9: SPECT/CT-Studies

SPECT/CT imaging was performed with a four-headed multiplexing multi-pinhole NanoSPECT (Bioscan Inc., Washington D.C.). Each head was outfitted with a tungsten collimator of nine 1.4 mm-diameter pinholes, imaging a cylindrical field of view that is 37 mm in diameter by 16 mm in length. The axial FOV is extended using a step-and-shoot helical scan of the animal, with the user defining a range from 16 to 180 mm according to the region to be imaged. The apertures used in this study provided a reconstructed resolution in the submillimetre range at 140 keV. The acquisition time per view was chosen for 1000 s. CT was performed with the integrated CT using a tube voltage of 45 kV and an exposure time of 1000 ms per view. After acquisition, SPECT and CT data were reconstructed iteratively with the HiSPECT software (Bioscan Inc., Washington D.C., USA) software. The SPECT and CT fusion was performed using the MIPtool software (version 1.20, Bioscan Inc.).

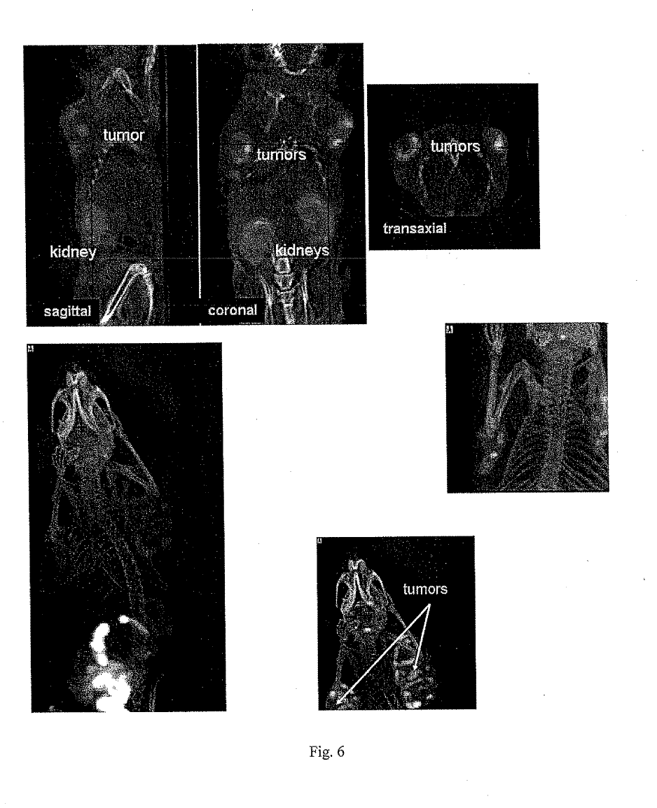

The results are shown in FIG. 6.

1. A compound of formula I 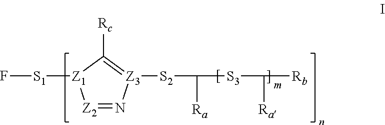
wherein
F is a folate or derivative thereof,
Z₁, Z₂, Z₃ are each, independently of each other C or N,
S₁, S₂, S₃ are each, independently of each other a single bond or a spacer,
Rₐ, R_{a′}, R_{b} are each, independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group,
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4, and
n is 1 or 2.

2. A compound according to claim 1, wherein said compound is of formula II or formula II′ 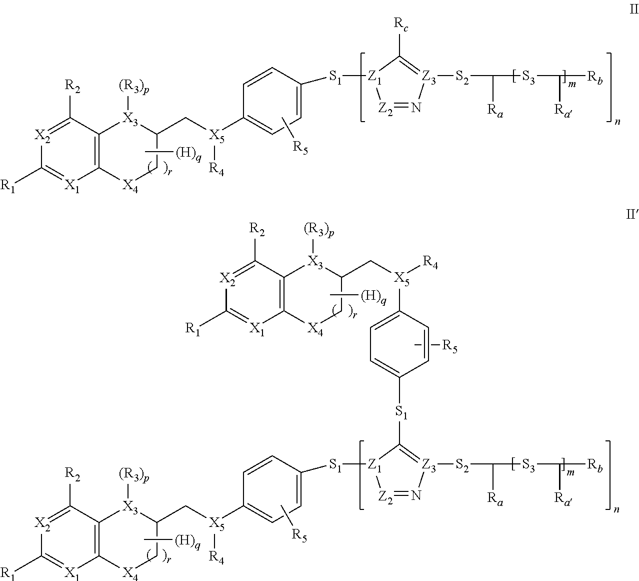
wherein
X₁, X₂, X₃, X₄ and X₅ are each, independently of each other C or N,
Z₁, Z₂, Z₃ are each, independently of each other C or N,
R₁ and R₂ are each, independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
S₁, S₂, S₃ are each, independently of each other a single bond or a spacer,
Rₐ, R_{a′}, R_{b} are each, independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group,
R_{c} is H, CO₂R′, COR′, —SO₃R′, —NHR′, or straight-chain or branched C1-C12 alkyl, which is unsubstituted or substituted by at least one CN, Hal, or NO₂, wherein R′ represents H, C1-C6 alkyl,
m is 0, 1, 2, 3 or 4,
n is 1 or 2,
p has a value of 0, 1 or 2,
q has a value of 1 to 7, and
r is 0 or 1.

3. A compound according to claim 1, wherein said compound is a compound selected from compounds of formulas IIa, IIb, IIc, IId and IIe 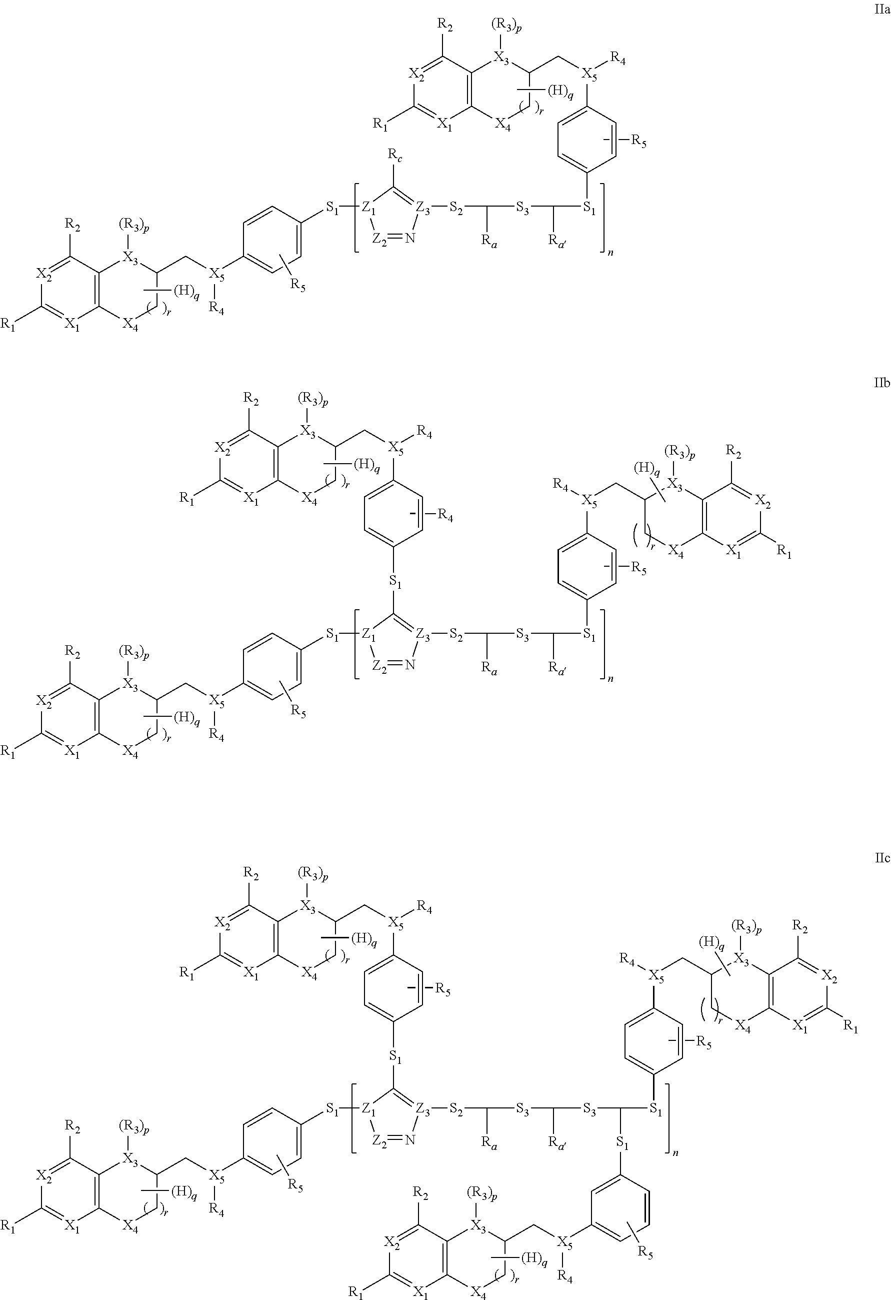 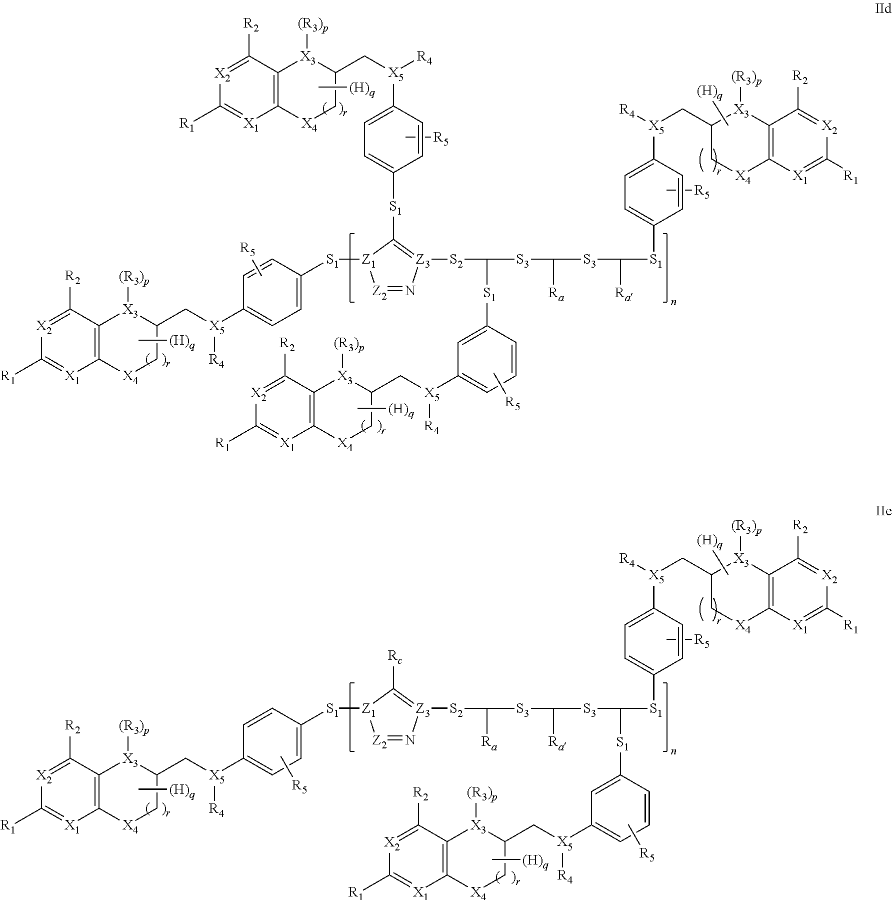
wherein
X₁, X₂, X₃, X₄ and X₅ are each, independently of each other C or N,
Z₁, Z₂, Z₃ are each, independently of each other, C or N,
R₁ and R₂ are each, independently of each other, H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
S₁, S₂, S₃ are each, independently of each other a single bond or a spacer,
Rₐ, R_{a′}, R_{b} are independently of each other —OH, —COOH, —NHR′, —CONH₂, —SH, a phosphine or a heterocyclic group, wherein R′ represents H, C1-C6 alkyl,
R_{c} is H, CO₂R′, COR′, —SO₃R′, —NHR′, or straight-chain or branched C1-C12 alkyl, which is unsubstituted or substituted by at least one CN, Hal, or NO₂, wherein R′ represents H, C1-C6 alkyl,
n is 1 or 2,
p has a value of 0, 1 or 2,
q has a value of 1 to 7, and
r is 0 or 1.

4. A compound according to claim 1, wherein S₁ is a single bond or straight-chain or branched C1-C12 alkyl, which is unsubstituted or substituted by at least one CN, Hal, OH, NH₂, SH, SO₃H or NO₂, and wherein one or more of the non-adjacent CH₂ groups may independently be replaced by —O—, —CO—, —CO—O—, —O—CO—, —NR′—, —NR′—CO—, —CO—NR′—, —CH═CH—, —C≡C—, or a five- or six-membered aromatic ring, which is unsubstituted or substituted with CN, Hal, NO₂, COR′ or COOR′, wherein R′ represents H or C1-C6 alkyl.

5. A compound according to claim 1, wherein S₂ and S₃ are each, independently of each other, a single bond or straight-chain or branched C1-C12 alkyl, which is unsubstituted or substituted by at least one CN, Hal, OH, NH₂ or NO₂, and wherein one or more of the non-adjacent CH₂ groups may independently be replaced by —O—, —CO—, —CO—O—, —NR′—, —NR′—CO—, wherein R′ represents H or C1-C6 alkyl.

6. A compound according to claim 1, wherein Z₁ is N, Z₃ is C and Z₂ is C or N.

7. A compound according to claim 1, wherein Z₁ is C and Z₂ and Z₃ are each N.

8. A compound according to claim 1, wherein m is 0 or m is 1.

9. A compound according to claim 1, wherein said compound is of formula III, 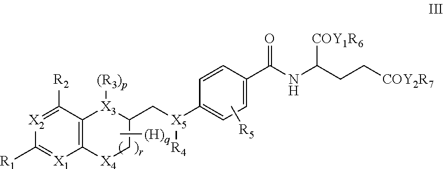
wherein
X₁, X₂, X₃, X₄ and X₅ are each, independently of each other C or N;
Y₁, Y₂ are each, independently of each other, C, O or N,
R₁ and R₂ are each, independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
p has a value of 0, 1 or 2,
q has a value of 1 to 7,
r is 0 or 1,
R₆ and R₇ are each, independently of each other H, straight chain or branched C₁-C₁₂ alkyl, which is unsubstituted or substituted by at least one CN, Hal or NO₂, or a group of formula IV 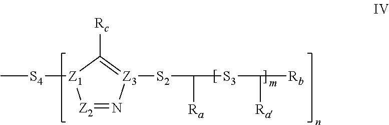
wherein
Z₁, Z₂, Z₃ are each, independently of each other C or N,
S₂, S₃, S₄ are each, independently of each other a single bond or a spacer,
Rₐ, R_{a′}, R_{b} are independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group,
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4, and
n is 1 or 2,
with the proviso that at least one of R₆ and R₇ is a group of formula IV.

10. A compound according to claim 9, wherein Z₁ is N, Z₃ is C and Z₂ is C or N.

11. A compound according to claim 9, wherein Z₁ is C and Z₂ and Z₃ are N.

12. A compound according to claim 9, wherein R₆ is H or straight chain or branched C₁-C₁₂ alkyl, which is unsubstituted or substituted by at least one CN, Hal or NO₂, and R₇ is a group of formula IV.

13. A compound according to claim 9, wherein R₆ is a group of formula IV, and R₇ is H or straight chain or branched C₁-C₁₂ alkyl, which is unsubstituted or substituted by at least one CN, Hal or NO₂.

14. A compound according to claim 9, wherein both R₆ and R₇ are each a group of formula IV.

15. A compound according to claims 9, wherein at least one of R₆ and R₇ is a group of formula IVa, IVb and/or a group of formula IVb′ 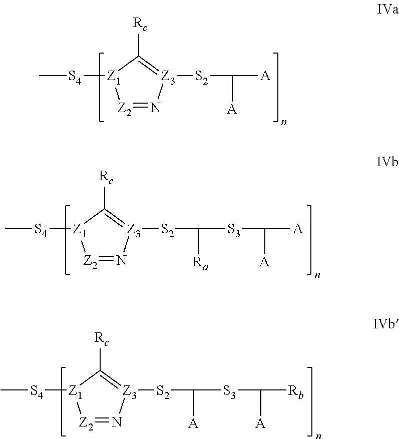
wherein
Z₁, Z₂, Z₃ are each, independently of each other, C or N,
S₂, S₃, S₄ are each, independently of each other, a single bond or a spacer,
A represents, in each case independently of each other, —COOH, —NH₂, —CONH₂, or —SH,
Rₐ, R_{b} are each, independently of each other, H, —OR′, —COOR′, —NHR′, —CONHR′, —SR′, a phosphine or a heterocyclic group, wherein R′ represents H or C1-C6 alkyl, or a F as defined hereinabove,
R_{c} is H, CO₂R′, COR′, —SO₃R′, or —NHR′, wherein R′ represents H, C1-C6 alkyl, or straight-chain or branched C1-C12 alkyl, which is unsubstituted or substituted by at least one CN, Hal, or NO₂, or a F as defined hereinabove, and
n is 1 or 2.

16. A compound according to claim 1, wherein said compound is selected from compounds of formulas V and V′, Va and Va′, Vb and Vb′ 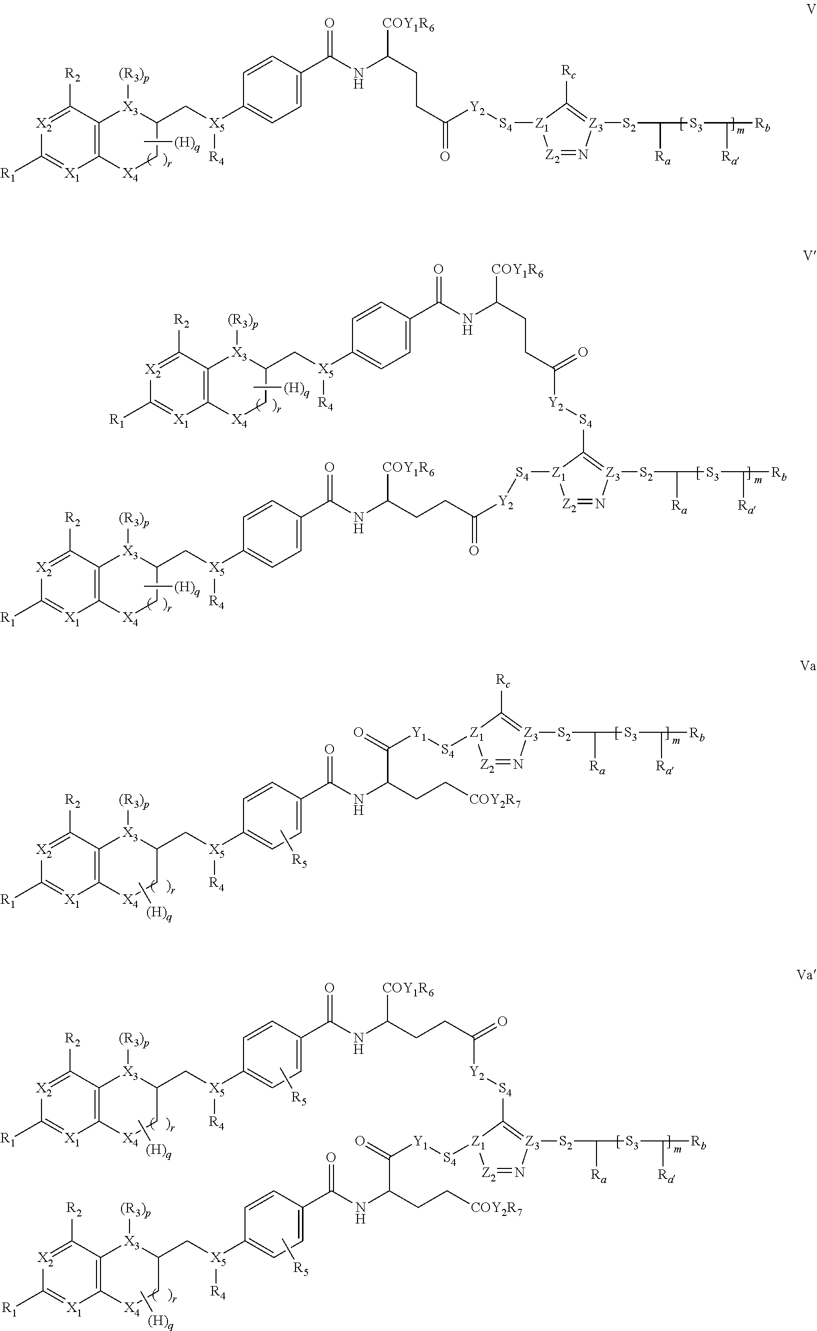 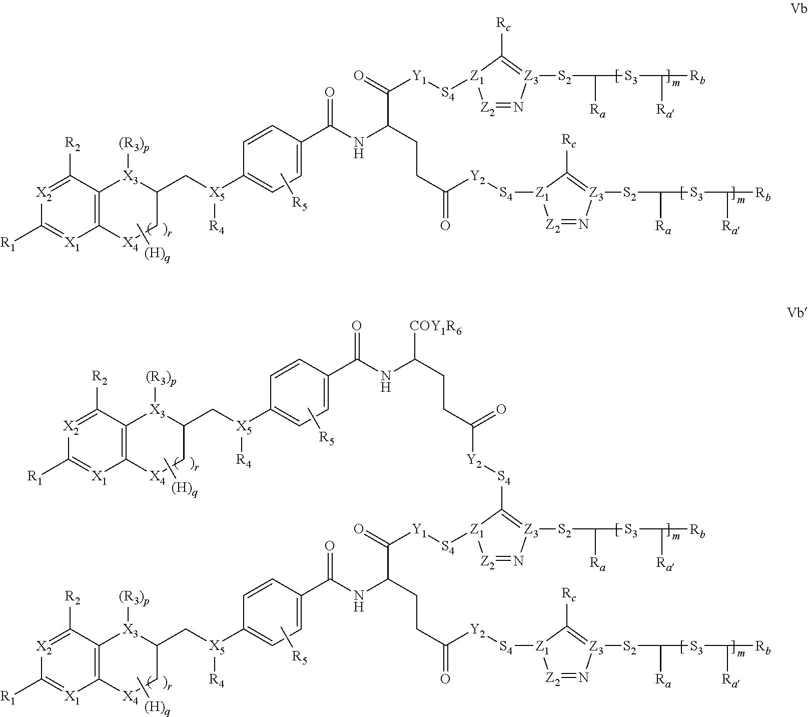
wherein
X₁, X₂, X₃, X₄ and X₅ are each, independently of each other, C or N;
Y₁, Y₂ are each, independently of each other, C, O or N,
Z₁, Z₂, Z₃ are each, independently of each other, C or N;
R₁ and R₂ are each, independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
R₆, R₇ are each, independently of each other, H or straight chain or branched C₁-C₁₂ alkyl, which is unsubstituted or substituted by at least one CN, Hal or NO₂,
S₂, S₃, S₄ are each, independently of each other a single bond or a spacer,
Rₐ, R_{a′}, R_{b} are each, independently of each other H, —OR′, —COOR′, —NHR′, —CONHR′, —SR′, a phosphine or a heterocyclic group, wherein R′ represents H or C1-C6 alkyl, or a F as defined hereinabove, and wherein of groups Rₐ, R_{a′} and R_{b} at least two adjacent groups are a donor group —OH, —COOH, —NHR′, —CONH₂, —SH, a phosphine or a heterocyclic group,
R_{c} is H, CO₂R′, COR′, —SO₃R′, —NHR′, wherein R′ represents H, C1-C6 alkyl, or straight-chain or branched C1-C12 alkyl, which is unsubstituted or substituted by at least one CN, Hal, or NO₂, or a F as defined hereinabove,
m is 0, 1, 2, 3, or 4,
p has a value of 0, 1 or 2,
q has a value of 1 to 7, and
r is 0 or 1.

17. A compound according to claim 16, wherein S₂, S₃, and S₄ are each, independently of each other straight-chain or branched C1-C8 alkyl, which is unsubstituted or substituted by at least one CN, Hal, OH, or NO₂ and wherein one or more of non-adjacent CH₂ groups may independently be replaced by —O—, —CO—, —CO—O—, —NR′—, —NR′—CO—, or —CO—NR′—, wherein R′ represents H or C1-C6 alkyl.

18. A compound according to claim 16, wherein m is 0.

19. A compound according to claim 16, wherein m is 1.

20. A compound according to claim 1, wherein said compound is a compound according to formulas VI, VI′, Via, VIa′, VIb or VIb′, 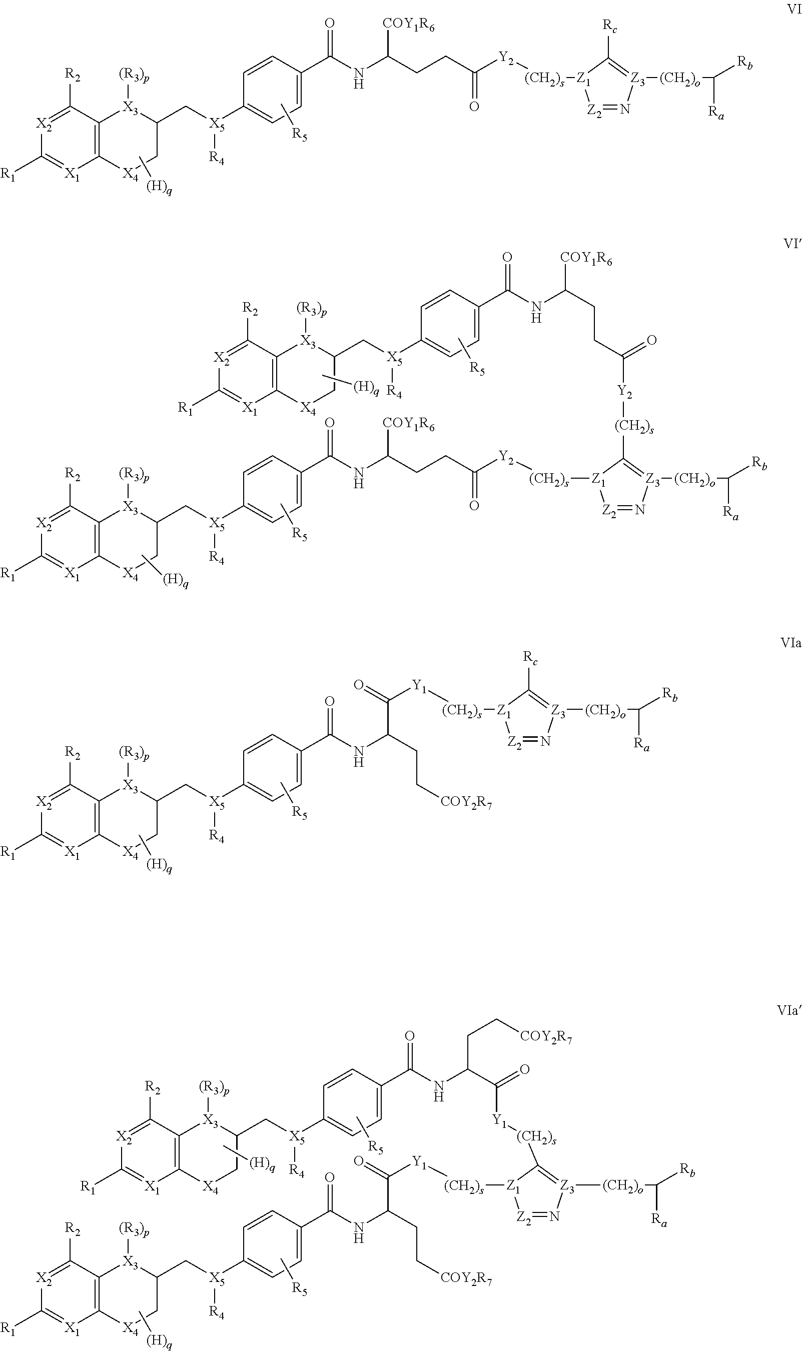 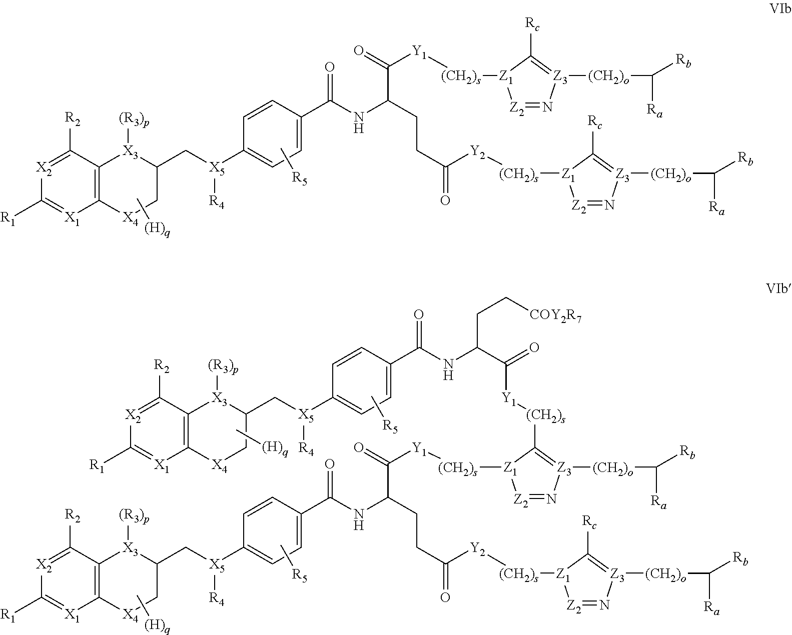
wherein
X₁, X₂, X₃, X₄ and X₅ are each, independently of each other, N or C,
Z₁, Z₂, Z₃ are each, independently of each other, C or N,
Y₁, Y₂ are each, independently of each other, C, O or N,
R₁ and R₂ are each, independently of each other H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other, H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
R₆, R₇ are each, independently of each other, H or straight chain or branched C₁-C₁₂ alkyl, which is unsubstituted or substituted by at least one CN, Hal or NO₂,
Rₐ, R_{b} are each, independently of each other, a donor group,
R_{c} is H, CO₂R′, COR′, —SO₃R′, or —NHR′, wherein R′ represents H or C1-C6 alkyl, or straight-chain or branched C1-C12 alkyl, which is unsubstituted or substituted by at least one CN, Hal, or NO₂,
p has a value of 0, 1 or 2,
q has a value of 1 to 7,
s is 1 to 8, and
o is 1 to 6.

21. A compound according to claim 20, wherein Z₁ is N, Z₃ is C and Z₂ is C or N.

22. A compound according to claim 20, wherein Z₁ is C and Z₂ and Z₃ are N.

23. A compound according to claim 1, wherein said compound is a compound according formulae VII, VII′, VIIa, VIIa′, VIIb, or VIIb′ 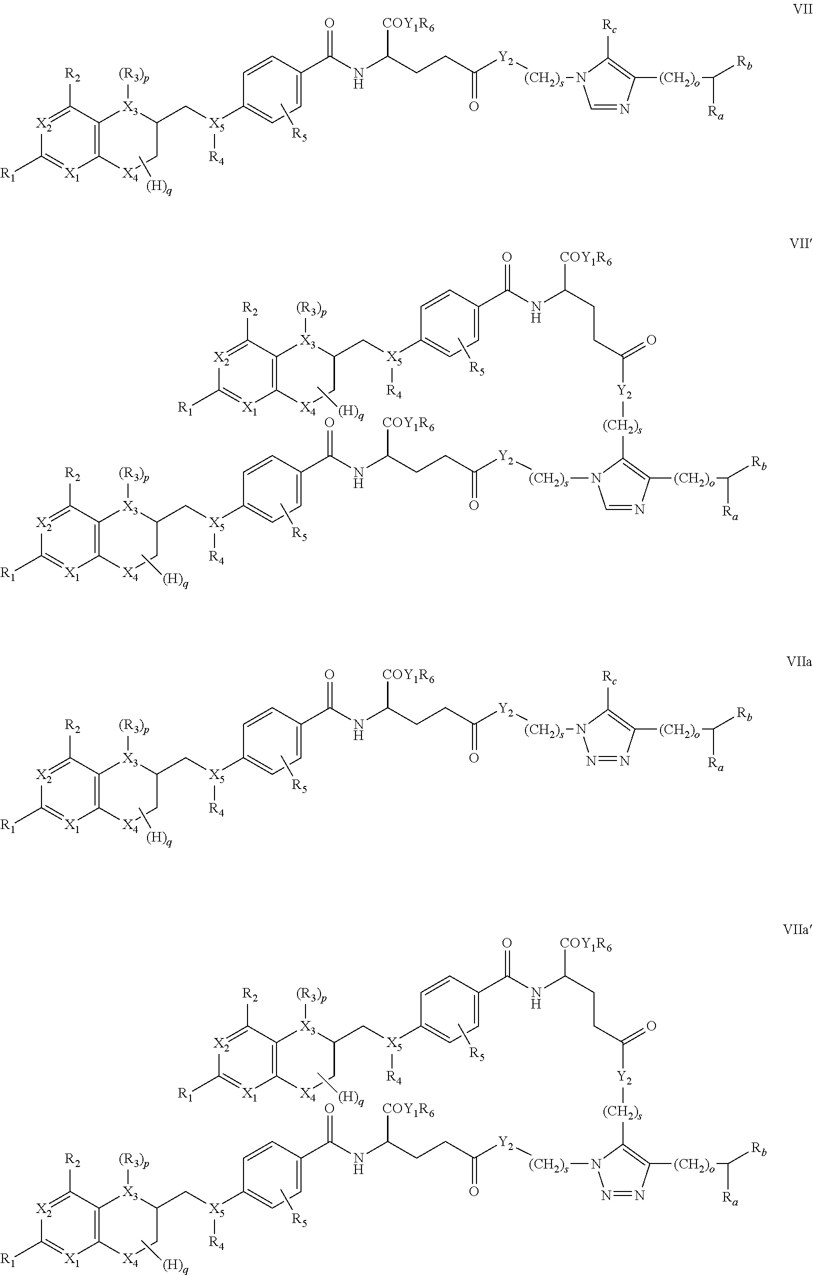 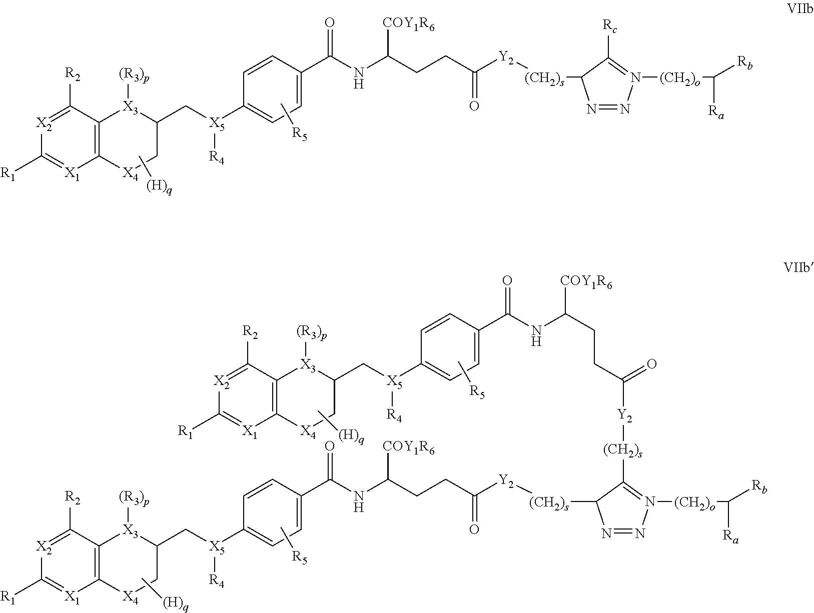
wherein
X₁, X₂, X₃, X₄ and X₅ are each, independently of each other, N or C,
Y₁, Y₂ are each, independently of each other, C, O or N,
R₁ and R₂ are each, independently of each other, H, Hal, —OR′, —NHR′, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl, wherein R′ is H or C1-C6 alkyl,
R₃ and R₄ are each, independently of each other, H, formyl, iminomethyl, nitroso, C1-C12 alkyl, C1-C12 alkoxy, C1-C12 alkanoyl, or halosubstituted C1-C12 alkanoyl,
R₅ is H, CN, Hal, NO₂, C1-C12 alkyl, C1-C12 alkoxy, C1-C12 alkanoyl, C2-C12 alkenyl, C2-C12 alkynyl, (C1-C12 alkoxy)carbonyl, or (C1-C12 alkylamino)carbonyl,
R₆ is H or straight chain or branched C₁-C₁₂ alkyl, which is unsubstituted or substituted by at least one CN, Hal or NO₂,
Rₐ and R_{b} are or independently of each other, a donor group,
R_{c} is H, CO₂R′, COR′, —SO₃R′, or —NHR′, wherein R′ represents H or C1-C6 alkyl, or straight-chain or branched C1-C12 alkyl, which is unsubstituted or substituted by at least one CN, Hal, or NO₂,
p has a value of 0, 1 or 2,
q has a value of 1 to 7,
s is 1 to 8, and
o is 1 to 6.

24. A compound according to claim 1, wherein R_{c} is H, CO₂R′, COR′, —SO₃R′, —NHR′ or C1-C12 alkyl, wherein R′ represents H or C1-C6 alkyl.

25. A compound according to claim 1, wherein Rₐ is —NH₂, R_{b} is —OH and R_{c} is H.

26. A compound according to claim 1, wherein R₆ is H or straight chain or branched C₁-C₁₂ alkyl.

27. A complex comprising a compound according to claim 1 and a radionuclide.

28. A complex according to claim 27, wherein the radionuclide is selected from ^{99m}Tc, ^{186/188}Re, ¹¹¹In⁺³, ^{67/68}Ga⁺³, ⁹⁰Y⁺³, ¹⁰⁹Pd⁺², ¹⁰⁵Rh⁺³, ¹⁷⁷Lu, ^{64/67}Cu ₁₆₆Ho, and ²¹³Bi.

29. A method of production of a compound according to claim 1, said method comprising: (i) synthesizing a heterocyclic ligand site for binding a radionuclide, (ii) linking said site through a suitable linker to a suitably protected pteroic or folic acid derivative and (iii) isolating the compound.

30. A method of production of a compound according to claim 1, said method comprising: (i) reacting an azido-derivatized folic acid with an ¹⁸F-labelled alkyne or alkyne substitute in a 1,3-cycloaddition and (ii) isolating the compound.

31. A method of production of a compound according to claim 1, said method comprising: (i) reacting a folic acid derivatized with an alkyne or alkyne substitute with an ¹⁸F-labelled azide in a 1,3-cycloaddition and (b) isolating the compound.

32. A method of production of a complex according to claim 27, said method comprising: reacting said compound with a radionuclide optionally in the presence of a reducing agent to form said complex.

33. A pharmaceutical composition comprising a diagnostic imaging amount or a therapeutically effective amount of at least one complex according to claim 27 and a pharmaceutically acceptable carrier therefor.

34. A method of using a complex according to claim 27 or a pharmaceutical composition comprising a complex of claim 27, said method comprising: preparing a diagnostic agent useful for convenient and effective administration to a subject in need for diagnostic imaging with said complex or pharmaceutical composition.

35. A method of using a complex according to claim 27 or a pharmaceutical composition comprising a complex of claim 27 comprising preparing a radiotherapeutic agent useful for convenient and effective administration to a subject in need for radiotherapy with said complex or pharmaceutical composition.

36. A method for diagnostic imaging of a cell or population of cells expressing a folate-receptor, said method comprising administering at least one complex according to claim 27 or a pharmaceutical composition comprising a complex of claim 27 in a diagnostic imaging amount, and obtaining a diagnostic image of said cell or population of cells.

37. A method for radiotherapy comprising administering to a subject in need thereof at least one complex according to claim 27, or a pharmaceutical composition comprising a complex of claim 27, in a therapeutically effective amount, and after localization of said at least one complex or composition in the desired tissues, subjecting the tissues to irradiation to achieve the desired therapeutic effect.

38. A method for in vitro detection of a cell expressing the folate receptor in a tissue sample, said method comprising contacting said tissue sample with a complex according to claim 27, or a pharmaceutical composition comprising a complex of claim 27, in an effective amount and for sufficient time and under sufficient conditions to allow binding to occur and detecting said binding by imaging techniques.

39. A single or multi-vial kit comprising, in one or separate vials, a compound according to claim 1, a source of a pharmaceutically acceptable reducing agent, and one or more optional additives.

40. A compound according to claim 1, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

41. A compound according to claim 2, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

42. A compound according to claim 3, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

43. A compound according to claim 9, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

44. A compound according to claim 15, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

45. A compound according to claim 16, wherein S₁, S₂, S₃ are independently of each other a single bond or a straight chain or branched C1-C12 alkyl, which is unsubstituted or substituted by at least one —CN, -Hal, —OH, —NH₂, —SH, —SO₃H or —NO₂, and wherein one or more of the non-adjacent CH₂ groups may independently be replaced by —O—, —CO—, —CO—O—, —O—CO—, —NR′—, —N═, —NR′—CO—, —CO—NR′—, —NR′—CO—O—, —O—CO—NR′—, —NR′—CO—NR′—, —CH═CH—, —C≡C—, —S—, —SO₃R′—, —PR′— or a five- or six-membered aromatic carbocyclic or heterocyclic ring, which is unsubstituted or substituted with —CN, -Hal, —NO₂, —COR′ or —COOR′, wherein R′ represents H or C1-C6 alkyl.

46. A compound according to claim 20, wherein Rₐ, R_{b} are each, independently of each other, a —OH, —COOH, —NHR′, —CONH₂, —SH, or a heterocyclic group selected from pyridyl, pyrrolyl, and thiazolyl, wherein R′ represents H or C1-C6 alkyl.

47. A compound according to claim 23, wherein Rₐ, R_{b} are each, independently of each other, a —OH, —COOH, —NHR′, —CONH₂, —SH, or a heterocyclic group selected from pyridyl, pyrrolyl, and thiazolyl, wherein R′ represents H or C1-C6 alkyl.
